(19) United States
(12) Patent Application Publication
(10) Pub. No.: US 2015/0353485 A1
(43) Pub. Date: Dec. 10, 2015

(54) SECURITY ELEMENT HAVING VOLUME HOLOGRAM AND PRINTED FEATURE

(71) Applicant: BAYER MATERIALSCIENCE AG, Monheim Am Rhein (DE)

(72) Inventors: Rainer Hagen, Leverkusen (DE); Thomas FÄCKE, Leverkusen (DE); Volker MARKER, Burscheid (DE); Horst BERNETH, Leverkusen (DE); Friedrich-Karl BRUDER, Krefeld (DE); Thomas RÖLLE, Leverkusen (DE); Marc-Stephan WEISER, Leverkusen (DE); Dennis HÖNEL, Zülpich-Wichterich (DE)

(21) Appl. No.: 14/762,256

(22) PCT Filed: Jan. 20, 2014
(86) PCT No.: PCT/EP2014/050987

§ 371 Date: Jul. 21, 2015

(30) Foreign Application Priority Data
Jan. 25, 2013 (EP) 13152715.2

(51) Int. Cl. C07C 309/17; C09D 11/03; C07C 215/40; G03H 1/00; C07C 211/63

(52) U.S. Cl. CPC C07C 309/17; G03H 1/0011; C07C 211/63; C07C 215/40; C09D 11/03

(57) ABSTRACT

The invention relates to a method for producing a security element having a holographic layer in which a hologram is arranged, characterized by at least the following steps: a) providing the holographic layer; b) exposing the holographic layer at least in sections via a master hologram to produce a hologram copy in the holographic layer; e) printing the holographic layer at least in sections with an ink, forming a printed feature, wherein the ink comprises the melt of a dye or a colorless component or a solvent and a dye dissolved therein or a colorless component dissolved therein; d) fixing the exposed holographic layer to produce the hologram in the holographic layer, wherein the printed feature and the hologram are arranged in the holographic layer such that the printed feature and the hologram overlap at least in sections. The invention further relates to a security feature which is produced or can be produced by said method.

Security printing today performs important functions in the authentication and identification of goods, merchandise and people. Security printing is employed, for example, on the packaging of technical products and consumer goods in order to characterize same. Printed devices offer protection against product piracy and help safeguard manufacturing chains. Security printing further serves an important function in protecting securities, banknotes, tax seals, ID cards and passports against manipulation and total forgery.

Plastics foils are particularly interesting, since they are flexible in use and convenient to integrate into manufacturing sequences and so are suitably combinable with security printing and further processable from reel or sheet into security labels, film tape, laminates and similar sheetlike products. The employment of plastics foils gives rise to new methods of reproduction and new products. The present application relates to such products.

The prior art discloses various methods of reproduction that are categorizable into printing, decorating and converting technologies. Printing and decorating are relevant to this application. Printing applies textual and graphical information atop or into plastics bodies. Existing methods of printing include, for example, inkjet printing, flexographic printing, offset lithography, gravure printing, laser printing, laser marking and also combinations thereof. Decorating is used to apply color, texture or graphics atop or into plastics bodies in order to enhance the esthetic value of the product. Existing methods used for decorative enhancement include electroplating, vacuum metallization, liquid coating, inkjet printing and various embossing techniques such as injection-compression molding, film embossing, colored embossing, relief embossing, hot embossing, hollow embossing and also combinations thereof.

New methods of reproduction, which are not widely disseminated and therefore are comparatively forgeryproof, include the printing/replicating of volume holograms via laser beam interference. Volume holograms belong to the class of diffractive optical variable image devices (DOVIDs). An overview of holography in practice is given by F. Unterseher et al. [Holography Handbook, Ross Books, 1982, ISBN 0894960164] and G. Saxby [“Practical Holography”, Third Edition, IOP, 2003, ISBN 075030912].

Volume holograms are usually executed as reflection holograms which, as their designation implies, become visible as a result of their reflecting incident light within the framework imposed by the defined holographic condition of diffraction. These holograms are wavelength selective and so the visual holograms can be reconstructed with white light. Multicolored volume reflection holograms, when compared with transmission holograms or particularly relief holograms, provide a true-color image across wide ranges of the viewing angle, as is the prerequisite for simple and hence confident authentication by the naked eye. Because it is possible to endow the holographic image with color, depth (to create 3D or 2D/3D effects) and animation (e.g., via multiplexed images which are separated via the viewing angle and observable via the parallactic motion), it may be both an overt security device and a decorative element.

Typical methods of recording and reconstructing multicolored volume reflection holograms have been known since at least 1970 and are described in the U.S. Pat. Nos. 3,532,406 and 4,959,284 for example. Typical recording materials for multicolored volume reflection holograms are photopolymers, see U.S. Pat. No. 4,963,471. Photopolymer holography is as the most important security printing technology for the coming years. In this application, the terms photopolymer hologram/photopolyrner holography are always associated with volume reflection holograms.

The prior art describes production protection labels comprising photopolymer holograms, for example in U.S. Pat. No. 7,268,926. The EP 1,892,587 and WO 2010/043403 applications additionally describe methods whereby the photopolymer hologram, which is executed as a primary (else overt or visible) device, is made still safer through additional partially or completely covert information. Methods to protect against manipulation at the label are known: the US 2003/0104155 application describes a layered construction consisting of substrate, volume hologram layer (e.g., photopolymer) and outer protective layer which is protected against deliberate manipulation, so the photopolymer cannot be bared and used as master for illegal laser contact copies. The solution described is a multilayered structure which ensures that the photopolymer layer comes apart in a defined manner when mechanically attacked. Serialization and individualization is also possible and known with product protection labels based on volume holograms and particularly photopolymer holograms: the EP 1,755,007 application describes a volume-holographic medium, for example a label, containing a machine-readable holographic bar code. This holographic serial information improves security of authentication over holographic labels without any serial information and similarly also over labels having a bar code which has been printed conventionally, for example via offset technology, and hence is simple to copy.

The trend, in summary, is thus in the direction of security products based on photopolymer holograms that (a) are authenticated via their primary security devices—defined herein as devices that are visible to the naked eye without further auxiliary means, (b) the primary security devices of which are protected against copying, forgery and manipulation, and that (c) offer additional protection against wholesale forgery through individual printed devices such as serial numbers, data particulars or similar product codes. The combination of requirements (a) to (c) currently offers the best precondition for secure authentication. The mass fabrication of such security products still poses challenges needing new solutions regarding the design of the primary devices and the efficiency of individualization. The prior art of industrial reproduction, i.e., mass fabrication of individualized photopolymer holograms, and the technical problem to be solved in relation thereto are elaborated in the sections which follow.

When individualized photopolymer holograms are to be mass produced and united in one production line, it is necessary to combine the replication unit [technology example see U.S. Pat. No. 6,824,929] with a digital hologram printer unit [technology example see EP patent No. 1,755,007 ]. Alternatively, there are color tuning processes for photopolymer holograms wherein the individual information is a false-color image which may be introduced into the photopolymer hologram subsequently and optionally also decentrally, away from the replication unit. The DE 10 2007 019 837 application describes such a holographic method of individualization in its elementary steps. Color tuning processes require not only specific adhesives and an adapted processing technology but also, in particular, complex systems and processes to locally cure the adhesives. Either approach—centralized as well as decentralized production—requires the deployment of holographic technologies that are costly and time-consuming to establish.

The technical problem addressed by the present application is therefore that of providing a photopolymer hologram security element that is simple to post-individualize via a conventional printing process. This individual printed image has to form an integral part within the security concept of the security element according to the present invention, so there is efficient protection against forgery, mass copying and duplication.

The object was solved by a method of producing a security element comprising a holographic layer containing a hologram, characterized by at least the steps of
a) providing the holographic layer;
b) exposing the holographic layer at least sectionwise via a master hologram to produce a hologram copy in the holographic layer;
c) printing the holographic layer at least sectionwise with an ink to form a printed device, wherein the ink comprises the melt of a dye or of a colorless component or a solvent and a dye or colorless component dissolved therein;
d) fixing the exposed holographic layer to produce the hologram in the holographic layer, wherein the printed device and the hologram are arranged in the holographic layer such that the printed device and the hologram overlap sectionwise at least.
The holographic layer and thhologram preferably carry the following main features:
The holographic layer consi s of a photopolymer aterial.
The holographic layer comprises a volume reflection hologram.
The hologram is designed as DOWD and therefore the primary security device,
The hologram is two- or poly-colored, i.e., it reconstructs light of two or more different wavelengths in the visible spectrum.
The holographic layer serves as substrate (carrier) for e individualized printed device.
The two devices, the holographic imaging information and the individualized printed graphic, are arranged atop each other regionwise at least. We refer to these as two design-integrated devices. This creates better protection against attempts to copy the lettered feature because the two devices cannot be simultaneously reproduced by a single method of reproduction. Two scenarios are offered for illustration: let us assume that the counterfeiter succeeds in using commercially available photocopying technology or digital camera technology to copy the printed device in satisfactory quality in terms of color and resolution. In this reproduction, however, the holographic imaging information will appear on the photographic copy not as an optically variable element but only as a colored blurring or as a shadow. Secondly, the printed device cannot be holographically copied in its original color: the attempt to produce a good contact copy by using known holographic methods of reproduction, as described in U.S. Pat. No. 6,824,929 for example, will cause the printed device to appear either invisible or as contrasted background should the printing color not scatter the wavelength of light used in the process or, when scattering does occur, be perceived in a color different from the original. [Excursion: This is known in the prior art, since the perception of colored printed images and of holograms which reflect defined ROB fractions of the ambient light back to the observer as “multicolored light”, is based on fundamentally different physical effects and depends differently on the external conditions of ambience and illumination.] In summary, the copy is easy to distinguish from the original in the two illustrative scenarios adduced because significant aspects of the image are absent or are reproduced incorrectly.

The printed device has the following further main properties and embodiments:
The printed device serves to individualize the security element.
The printed device is a security feature in that the liquid printing ink penetrates into the substrate (i.e., the holographic layer) and thus forms a manipulation-proof integral constituent thereof. The security function results from the fact that the hologram is not simple to isolate from the individual printed device and thus used directly as a master for illegal mass copying.
The printed device is a security device in that it alters the imaging hologram. The migration of the liquid printing ink into the substrate exhibits an interaction with the hologram such that the grating structures of the hologram undergo swelling, with the effect that the reconstruction color of the hologram and/or its diffraction efficiency and/or its reconstruction angle (the eyebox) become irreversibly altered as a consequence of the migration of the constituents of the liquid ink. Contact copies of the hologram thus always bear an individual hallmark even if co-copying of the printed device itself as an additional hologram is successfully avoided. Product recognition systems as offered today by the security industry could be used to recognize, and trace back, such an illegally copied code.
The printed device is produced using conventional liquid ink printing processes, such as inkjet printing, thermal transfer printing or thermal diffusion printing. These processes are established. They are predestined for the printing of variable data, such as serial numbers and the like, up to large numbers of pieces, since the leadtime on changing over the printed image is minimal. Further advantages reside in the simplicity of adaptation to existing manufacturing processes, the simple handling, the flexibility (liquid inks and printing parameters are conformable to the requirements of the printing substrate), the good to very good quality of printing, the ease of maintenance and the low noise.
An inkjet printer, once the ideal conditions for printing have been determined, is an efficient and consistent means for lettering. The specific advantage of inkjet printing for the purposes of this application is that the liquid ink and the substrate can be developed and mutually adjusted such that the printed device becomes an integrated security device having the abovernentioned properties.

The liquid printing ink is notable for the following properties:
It consists of two or more individual components.
Component 1 is an active substance notable for good solubility in the photopolymer film used as printing substrate.
Component 2 is a solvent for component 1.

The components are selected such that the following properties can be conformed to the requirements:
Viscosity
Migration rate into the photopolymer film
Resistance of printed image, for example to water, light (UVIVIS/TR), abrasion and chemicals
High achievable resolution, e,g., 8 to 24 dots/mm (200 to 600 dpi)

Component 1 may be
a) a dye which absorbs in the UV, VIS and/or IR range, preferably in the visible spectrum. The effect rests essentially but not exclusively on a direct visualization by absorption or scattering. The possible second effect is to change the properties of the hologram.
b) a colorless substance, the effect of which rests exclusively on a change to the properties of the hologram.

It is also possible to use two or more components of type (a) or of type (b) or mixtures of (a) and (b).

Component 2 is preferably compatible with inkjet printing as regards volatility and viscosity, After printing, it evaporates, i.e., does not remain for good in the substrate.

Measures to fix the printed liquid inks are: subsequent application of a material by printing, pouring, dipping or spraying. Two effects here are alternatively responsible to fix the liquid ink at molecular level:
attachment to comparatively high molecular weight chemical by covalent or ionic bonding
conversion into a less soluble molecular component

Further properties of the security element come to bear in specific embodiments;
The hologram is full-colored. A full-colored hologram concept requires three or more primary colors. Further colors make it possible to achieve a higher gamut, i.e., to construct a larger color space, meeting even higher color design requirements.
Full color offers higher protection against illegal contact copies because two or more mutually adjusted laser contact exposures are needed in order that the entire color spectrum may appear in the copy as well. The same holds for the emulation of the hologram. The reconstruction wavelengths of the hologram, when viewed as a contribution to copying protection, are preferably located in ranges which are not covered by industrially available holography-capable lasers, for the purpose of avoiding the case where the counterfeiter gains access to the full set of lasers/laser wavelengths which is needed to copy the hologram in its full color. Known laser wavelengths which preferably do not coincide with the reconstruction wavelengths of the hologram are: a) 488, 514, 532, 568, 633, and 647 nm; b) 647, 671 and 694 nm; c) 413, 442, 458 and 476 nm. Wavelengths of the (a) category listed are those of bright, conspicuous colors, which are of particular value for the primary holographic security device and thus are vitally crucial to achieve the abovementioned purpose. The wavelengths of reds and blues are listed under (b) and (c) respectively, and they are each located at the edge of the visible spectrum and therefore number among the less brilliant hologram colors. Both are accordingly of secondary significance.
The hologram is spatial, i.e., it reproduces imaging information in true 3D or at least depth-resolved imaging planes, i.e., 2D/3D devices.
The hologram carries covert devices which only become visible on appropriate illumination or to means other than the naked eye.
The hologram has a restricted solid angle range in which it reconstructs, so there are viewing directions whence the imaging information is not visible. To wit, there is a solid angle range whereinto the hologram does not refract light, in front of the security element. One prerequisite for the printed device to be recognizable by a machine is accordingly established.
An advantage with regard to the anti-counterfeit security provided by the security element is accurate registration of hologram and printed device in the hologram layer. [Excursion: Registration in printing refers to the vertical alignment of the individual colors in multicolored printing. Registration refers in all printing processes to the properly positioned printing in two or more successive printing operations. Herein we use the term for successive printing operations which correlate the two different printing technologies into one printed image.] The individual devices in the combined security device are in accurate registration and their graphical structures cooperate such that they form one graphical overall representation. The overall printed image is neither blurred nor fuzzy and free of color shifts with a quality-reducing effect. The two devices may be mutually complementary for example. One example thereof is a line pattern similar to a Guilloche pattern wherein one device represents one part of the pattern and the second device, the remaining part. Alternatively, imaging parts of the two devices may be overlapping. The examples adduced are for illustration and are not to be construed as narrowing the broad claim to possible designer-created manifestations of the security device.
The printing units must accordingly be equipped with dedicated positioning and pressing means to ensure the required accuracy of positioning. Roll-fed printing presses today come with automatic control of registration, known as in-line color registration measurement. When the marks are not exactly aligned, automatic correction is applied to the printing units. To produce the security element of the present invention, accurate registration is preferably effected via two types of markers with corresponding measuring means: 1.) markers which are part of the inkjet-printed image of the present invention. 2.) markers which as part of the hologram master are co-transferred into the hologram copy. Corresponding measuring means capture the two different types of marker. In order that the ready-produced security element is left uncut, the marks are preferably situated within the printed image and are engineered such that they are scarcely visible under ambient illumination, if at all. The problem is solved in the case of the hologram by preferably using a hologram marker which lies very deeply behind the copying film plane, more preferably a long way in front of the copying film plane and which becomes visible under punctuate monochromatic light as has to be used in the sensor, but which becomes blurred under ambient light to such an extent that it can no longer be recognized as an image. This effect is an intrinsic presence in the case of volume reflection holograms which are sufficiently far outside the film plane. This distance is from 0.5 to 100 cm, preferably from 1.0 to 20 cm, more preferably from 1.0 to 10 cm. The issue is resolved in the case of inkjet printing by making marks having a diameter of 0.1 to 2.0 mm, preferably about 1 mm, very small, so they are scarcely perceivable any longer. Alternatively, the markers are also introducible very close to, but not into the predefined area of the security device, so the loss on cutting can be kept to a minimum.
The printed device is a visible alphanumeric code.
The printed device is a 2D or 3D bar code.
The printed device is a digital portrait of a person.
The printed device is machine-readable.
The substrate consisting of or containing a sheetlike construct, which is referred to as the holographic copying film and which is photopolymer-based, is also the printing substrate.
The printing substrate is preferably between 1 and 65 μrn, preferably between 5 and 20 μm and more preferably between 10 and 17 μm in thickness.
The printing substrate may itself be applied to a carrier foil.
The carrier foil consist of plastic or paper, preferably of plastic, more preferably of transparent, nonscattering plastic.
When photopolymer and carrier foil are transparent, the security element can serve as decorative element if it is applied to a surface, for example a product package or a casing. The printed image or the color of the product package is not covered entirely, but remains visible, at least partially. It is particularly preferable for the overall graphics of the security element to be aligned to the graphics of the underlying surface, so the design of the security element augments the packaging or casing design.
The security element may have, on the printed side of the hologram layer, a covering foil or a covering varnish which has to be applied after printing.
The security element may comprise two or more carrier foils and covering foils/varnishes.

The security element is produced in a plurality of steps:

| | |
|---|---|
| 1st | Providing the holographic film. |
| 2nd | Providing the holographic master. |
| 3rd | Providing the liquid printing ink. |
| 4th | Holographic exposure sequence. |
| 5th | Fixing the hologram and bleaching the holographic film. |
| 6th | Individual printing. |
| 7th | Converting (optional). |

The term converting subsumes process steps to finish the security element and hence to prepare for subsequent process steps. Typical converting steps are: applying a covering varnish, delamination or relamination of laminates and substrates, die-cutting, embossing, laminating with a transfer foil or applying a layer of adhesive.

Printing (6) can take place before and/or after the exposure sequence (4). Printing (6) can take place before andlor after the fixing step (5). Printing can take place before or after the converting steps (7), provided as will he appreciated that the printing side of the printing substrate is not covered during the printing step.

It is preferable for the printing (6) to take place as the last process step or at least after the fixing step (5). This subsequent individualization of the readied security element permits decentralized characterization, for example in the labeling unit or in the manufacture of a security document. Characterization includes individualization, personalization, serialization and all forms of recordation/signoff, for example by printing with a digitalized device to be authenticated.

The security element of the present invention may be transferredlintegrated into for example a product protection label or brand label or . The security element is similarly useful for certification of ID cards, travel passports, credit cards, etc. In the case of ID cards, the label is preferably transferred to the data page and then securely integrated into the transparent frontpage masking (front laminate or covering varnish). It may alternatively remain at the surface as batch. The security element may further be integrated into a banknote as batch, thread or strip. The position in the banknote is free. Conceivable possibilities are sticking on, weaving in or integration as an optical window or element of a window.

In one preferred manifestation of the method according to the present invention, the printed device is formed before and/or after the production of the hologram copy and/or the fixing of the exposed holographic layer, wherein the printed device is preferably formed after the production of the hologram copy, more preferably after the fixing of the exposed holographic layer. The liquid ink contains colored and/or colorless, in particular salt-type, components and also a solvent. The liquid ink more particularly contains no constituents that are insoluble in the solvent.

In one preferred embodiment of the method according to the present invert ion, the colored component of the liquid ink is a salt-type dye, more particularly selected from cationic dyes which preferably belong to the following classes: acridine dyes, xanthene dyes, thioxanthene dyes, phenazine dyes, phenoxazine dyes, phenothiazine dyes, coumarin dyes, tri(het)arylmethane dyes, in particular diamino-and triamino (het)arylmethane dyes, mono-, di-, tri-, tetra- and pentamethinecyanine dyes, hemicyanine dyes, diazahemicyanine dyes, zeromethine dyes, in particular naphthola.ctam dyes, streptocyanine dyes, externally cationic merocyanine dyes, externally cationic neutrocyanine dyes, externally cationic phthalocyanine dyes, externally cationic anthraquinone dyes, externally cationic azo dyes, or anionic dyes which preferably belong to the following classes: oxonols, di- and trihydroxytriarylmethane dyes, the group of merocyanine, neutrocyanine, coumarin, anthraquinone, anthrapyridone, dioxazine, mono-,dis- and trisazo dyes having at least one sulfo group, the group of acridine, xanthene, thioxanthene, phenazine, phenoxazine, phenothiazine, tri(het)arylinethane dyes, in particular diamino- and triamino(het)aryltriethane dyes, having at least two sulfo groups, phthalocyanines and azo metal complexes bearing at least one sulfo group, and also mixtures thereof.

In a likewise preferred embodiment of the method according to the present invention, the colorless component of the liquid ink provided is a salt-type substance more particularly selected from colorless salts such as ammonium, sulfonium, phosphonium, cycloammonium or cycloimmonium salts of organic mono-, his- or trissulfonic acids, wherein both the cation and the anion each bear at least one long-chain, optionally branched alkyl moiety, cationic or anionic whiteners or anionic complexes of rare earths.

In a likewise preferred embodiment of the method according to the present invention, the liquid ink is a mixture of at least one salt-type dye and/or at least one colorless component which is a salt-type substance.

In a further preferred embodiment of the method according to the present invention, the dye and/or the colorless component migrates into the holographic layer, wherein particularly the grating structure of the hologram copy and/or of the hologram is swollen by the dye which migrates into the holographic layer. Preferably, the reconstruction color of the hologram, its diffraction efficiency and/or reconstruction angle are irreversibly altered by the dye which migrates into the holographic layer.

The dye used is preferably chosen such that this reflects white light in the visible wavelength range, in particular in the range from 400 to 800 nm.

In a further preferred embodiment, the holographic layer comprises or consists of a photopolymer material and/or the holographic layer is on a carrier. Furthermore, the hologram may be formed by a volume hologram, in particular by a volume reflection hologram, sectionwise at least. Advantageously, the hologram reconstructs light of at least two different wavelengths in the visible spectrum, wherein the different wavelengths are more particularly at least 10 nm, preferably at least 20 nm or even 30 nm apart. As a result, even the human eye becomes capable of perceiving various hues, which further enhances anti-counterfeit security.

The invention provides for at least sectionwise printing of the holographic layer with a liquid ink to form the printed device. Preferably, the hologram area overprinted by the printed device comprises from 5 to 95% of the entire area of the hologram, in particular from 10 to 90%. It is further advantageous in this case when the printed device projects beyond the hologram on one side at least.

In a further preferred manifestation, the printed device is an image, a pattern, an alphanumeric code, a 2D or 3D bar code or some other machine-readable code, such as a biometric feature, wherein the resolution and content comprises in particular sufficiently precisely defined information for the software-based image recognition by machine, and/or product- or person-related information designed as a security feature which is covert or only recognizable via ancillary means.

The method of the present invention is in principle performable using any suitable printing procedure. Inkjet printing is particularly preferable.

The present invention further provides a security element obtainable or obtained by a method according to the present invention.

The invention additionally provides a document, a certificate or other document of value, a banknote, an ID card, a high security access card, a tax seal, an electronic ticket, an electronic card, a credit card, a cashcard or a product package or product label for consumer durables, industrial goods and consumable goods, endowed with a security element as claimed in claim 14.

The invention further provides the method of using an ink to improve the anti-counterfeit security of a hologram wherein the ink comprises the melt of a dye or of a colorless component or a solvent and a dye or colorless component dissolved therein. Any desired combinations are also possible.

The chemical makeup of the photopolymer-based printing substrate will now be described.

Polyisocyanate component a) may be any compounds well known per se to a person skilled in the art, or mixtures thereof, which on average have two or more NCO functions per molecule. These may have an aromatic, an araliphatic, an aliphatic or a cycloaliphatic base. Monoisocyanates and/or unsaturated polyisocyanates may also be used in minor amounts.

Suitable candidates include, for example, butylenes diisocyanate, hexamethylene diisocyanate (FIDE), isophorone diisocyanate (IPDI), 1,8-diisocyanato-4-(isocyanatomethyl)octane, 2,2,4- and/or 2,4,4-trimethylhexamethylene diisocyanate, the isomeric bis(4,4′-isocyanatocyclohexyl)methane and their mixtures of any desired isomeric content, isocyanatomethyl-1,8-octane diisocyanate, 1,4-cyclohexylene diisocyanate, the isomeric cyclohexanedimethylene diisocyanates, 1,4-phenylene diisocyanate, 2,4- and/or 2,6-tolylene diisocyanate, 1,5-naphthylene diisocyanate, 2,4′- or 4,4′-diphenyltnethane diisocyanate and/or triphenylmethane 4,4′, 4″-triisocyanate.

It is similarly possible to use derivatives of monomeric di- or triisocyanates having urethane, urea, carbodiimide, acylurea, isocyanurate, allophanate, biuret, oxadiazinetrione, uretdione and/or iminooxadiazinedione structures.

Preference is given to the use of polyisocyanates based on aliphatic and/or cycloaliphatic di- or triisocyanates.

It is particularly preferable for the polyisocyanates of component a) to be di- or oligomerized aliphatic and/or cycloaliphatic di- or triisocyanates.

Very particular preference is given to isocyanurates, uretdiones and/or iminooxadiazinediones based on HDI, 1,8-diisocyanato-4-(isocyanatomethypoctane or mixtures thereof.

Component a) may likewise utilize NCO-functional prepolymers having urethane, allophanate, biuret and/or amide groups. Prepolymers of component a) are obtained in a conventional manner by reacting monomeric, oligomeric or polyisocyanates a1) with isocyanate-reactive compounds a2) in suitable stoichiometry in the presence or absence of catalysts and solvents.

Polyisocyanates a1) may be any aliphatic, cycloaliphatic, aromatic or araliphatic di- and triisocyanates known per se to a person skilled in the art, it being immaterial whether they were obtained by phosgenation or by phosgene-free processes. In addition, it is also possible to use the conventional higher molecular weight descendant products, of monomeric di- and/or triisocyanates having a urethane, urea, carbodiimide, acylurea., isocyanurate, allophanate, biuret, exadiazinetrione, uretdione or iminooxadiazinedione structure each individually or in any desired mixtures thereamong.

Examples of suitable monomeric di- or triisocyanates useful as component al) include butylene diisocyanate, hexamethylene diisocyanate (HDI), isophorone diisocyanate (IPDI), trimethylhexamethylene diisocyanate (TMDI), 1,8-diisocyanato-4-(isocyanatomethyl)octane, isocyanatornethyl-1,8-octane diisocyanate (TIN), 2,4- and/or 2,6-tolylene diisocyanate.

Isocyanate-reactive compounds a2) for constructing the prepolymers are preferably OH-functional compounds. These are analogous to the OH-functional compounds described hereinbelow for component b).

The use of amines for prepolymer preparation is also possible. For example, ethylenediamine, diethylenetriarnine, triethylenetetramine, propylenediamine, diaminocyclohexane, diaminobenzene, diaminobisphenyl, difunctional polyamines, such as, for example, the Jeffamine® amine-terminated polymers having number average molar masses of up to 10 000 g/mol and any desired mixtures thereof with one another are suitable.

For the preparation of prepolymers containing biuret groups, isocyanate is reacted in excess with amine, a biuret group forming. All oligomeric or polymeric, primary or secondary, difunctional amines of the abovernentioned type are suitable as amines in this case for the reaction with the di-, tri- and polyisocyanates mentioned.

Preferred prepolymers are urethanes, allophanates or biurets obtained from aliphatic isocyanate-functional compounds and oligomeric or polymeric isocyanate-reactive compounds having number average molar masses of 200 to 10 000 g/mol; particular preference is given to urethanes, allophanates or biurets obtained from aliphatic isocyanate-functional compounds and oligomeric or polymeric polyols or polyamines having number average molar masses of 500 to 8500 g/mol. Very particular preference is given to allophanates formed from HDI or TMDI and difunctional polyetherpolyols having number average molar masses of 1000 to 8200 g/mol.

The prepolymers described above preferably have residual contents of free monomeric isocyanate of less than 1% by weight, particularly preferably less than 0.5% by weight, very particularly preferably less than 0.2% by weight.

In addition to the prepolymers described, the polyisocyanate component can of course contain further isocyanate components proportionately. Aromatic, araliphatic, aliphatic and cycloaliphatic di-, tri- or polyisocyanates are suitable for this purpose. It is also possible to use mixtures of such di-, tri- or polyisocyanates, Examples of suitable di-, tri- or polyisocyanates are butylene diisocyanate, hexamethylene diisocyanate (HDI), isophorone diisocyanate 1,8-dlisocyanato-4-(isocyanatomethyl)octane, 2,2,4- and/or 2,4,4-trimethylhexamethylene diisocyanate (TMDI), the isomeric bis(4,4′-isocyanatocyclohexyl)methanes and mixtures thereof having any desired isomer content, isocyanatomethyl-1,8-octane diisocyanate, 1,4-cyclohexylene diisocyanate, the isomeric cyclohexanedimethylene diisocyanates, 1,4-phenylene diisocyanate, 2,4- and/or 2,6-tolylene diisocyanate, 1,5-naphthylene diisocyanate, 2,4′- or 4,4′-diphenylmethane diisocyanate, triphenylmethane 4,4′,4 ″-triisocyanate or derivatives thereof having a urethane, urea, carbodiimide, acylurea, isocyanurate, allophanate, biuret, oxadiazinetrione, uretdione or iminooxadiazinedione structure and mixtures thereof. Preference is given to polyisocyanates based on oligomerized and/or derivatized diisocyanates which were freed from excess diisocyanate by suitable processes, in particular those of hexamethylenediisocyanate. The oligorneric isocyanurates, uretdiones and iminooxadiazinediones of HDI and mixtures thereof are particularly preferred.

It is optionally also possible for the polyisocyanate component a) proportionately to contain isocyanates which are partially reacted with isocyanate-reactive ethylenically unsaturated compounds. αβ-Unsaturated carboxylic acid derivatives, such as acrylates, rnethacrylates, maleates, fumarates, maleimides, acrylamides, and vinyl ethers, propenyl ethers, allyl ethers and compounds which contain dicyclopentadienyl units and have at least one group reactive towards isocyanates are preferably used here as isocyanate-reactive ethylenically unsaturated compounds; these are particularly preferably acrylates and methacrylates having at least one isocyanate-reaetive aroup. Suitable hydroxy-functional acrylates or rriethacrylates are, for example, compounds such as 2-hydroxyethyl(meth)acrylate, polyethylene oxide mono(meth)acrylates, polypropylene oxide mono(meth)acrylates, polyalkylene oxide mono(meth)acrylates, poly(ε-caprolactone) mono(meth)acrylates, such as, for example, Tone® M100 (Dow, USA), 2-hydroxypropyl(meth)acrylate, 4-hydroxybutyl(meth)acrylate, 3-hydroxy-2,2-dimethylpropyl(meth)acrylate, the hydroxy-functional mono-, di- or tetra(meth)acrylates of polyhydric alcohols, such as trimethylolpropane, glycerol, pentaerythritol, dipentaerythritol, ethoxylated, propoxylated or alkoxylated trimethylolpropane, glycerol, pentaerythritol, dipentaerythritol and industrial mixtures thereof. In addition, isocyanate-reactive oligomeric or polymeric unsaturated compounds containing acrylate and/or methacrylate groups, alone or in combination with the abovementioned monomeric compounds, are suitable. The proportion of isocyanates which are partly reacted with isocyanate-reactive ethylenically unsaturated compounds, based on the isocyanate component a), is 0 to 99%, preferably 0 to 50%, particularly preferably 0 to 25% and very particularly preferably 0 to 15%.

It may also be possible for the abovementioned polyisocyanate component a) to contain, completely or proportionately, isocyanates which are reacted completely or partially with blocking agents known to the person skilled in the art from coating technology. The following may be mentioned as an example of blocking agents: alcohols, lactams, oximes, malonic esters, alkyl acetoacetates, triazoles, phenols, imidazoles, pyrazoles and amines, such as, for example, butanone oxime, diisopropylamine, 1,2,4-triazole, dimethyl-1,2,4-triazole, imidazole, diethyl malonate, ethyl acetoacetate, acetone oxime, 3,5-dimethylpyrazole, ε-caprolactam, N-tert-butylbenzylamine, cyclopentanone carboxyethyl ester or any desired mixtures of these blocking agents.

It is particularly preferable for the polyisocyariate component to be an aliphatic polyisocyariate or an aliphatic prepolymer and preferably an aliphatic polyisocyanate or a prepolymer having primary NCO groups.

Any polyfunctional, isocyanate-reactive compounds which have on average at least 1.5 isocyanate-reactive groups per molecule can be used in principle as polyol component b).

In the context of the present invention, isocyanate-reactive groups are preferably hydroxyl, amino or thio groups, and hydroxy compounds are particularly preferred.

Suitable polyfunctional, isocyanate-reactive compounds are, for example, polyester-, polyether-, polycarbonate-, poly(meth)acrylate- and/or polyurethanepolyols.

Suitable polyester polyols are, for example, linear polyester diols or branched polyester polyols, as are obtained in a known manner from aliphatic, cycloaliphatic or aromatic di- or polycarboxylic acids or their anhydrides with polyhydric alcohols having an OH functionality of ≧2.

Examples of such di- or polycarboxylic acids or anhydrides are succinic, glutaric, adipic, pimelic, suberic, azelaic, sebacic, nonanedicarboxylic, decanedicarboxylic, terephthalic, isophthalic, o-phthalic, tetrahydrophthalic, hexahydrophthalic or trimellitic acid and acid anhydrides, such as o-phthalic, trimellitic or succinic anhydride or any desired mixtures thereof with one another.

Examples of suitable alcohols are ethanediol, di-, tri- or tetraethylene glycol, 1,2-propanediol, di-, tri- or tetrapropylene glycol, 1,3-propanediol, 1,4-butanediol, 1,3-butanediol, 2,3-butanediol, 1,5-pentanediol, ,6-hexanediol, 2,2-dimethyl-1,3-propanediol, 1,4-dihydroxycyclohexane, 1,4-dimethylolcyclohexane, 1,8-octanediol, 1,10-decanediol, 1,12-dodecanediol, trimethylolpropane, glycerol or any desired mixtures thereof with one another.

The polyester polyols may also be based on natural raw materials, such as castor oil. It is also possible for the polyester polyols to be based on homo- or copolymers of lactones, as can preferably be obtained by an addition reaction of lactones or lactone mixtures, such as butyrolactone, ε-caprolactone and/or methyl-ε-caprolactone, with hydroxy-functional compounds, such as polyhydric alcohols having an OH functionality of ≧2 for example of the aforementioned type.

Such polyester polyols preferably have number average molar masses of 400 to 4000 g/mol, particularly preferably of 500 to 2000 g/mol. Their OH functionality is preferably 1.5 to 3.5, particularly preferably 1.8 to 3.0.

Suitable polycarbonate polyols are obtainable in a manner known per se by reacting organic carbonates or phosgene with diols or diol mixtures.

Suitable organic carbonates are dimethyl, diethyl and diphenyl carbonate.

Suitable diols or mixtures comprise the polyhydric alcohols mentioned in connection with the polyester segments and having an OH functionality of ≧2, preferably 1,4-butanediol, 1,6-hexanediol andlor 3-methylpentanediol, or else polyester polyols can be converted into polycarbonate polyols.

Such polycarbonate polyols preferably have number average molar masses of 400 to 4000 g/mol, particularly preferably of 500 to 2000 g/mol. The OH functionality of these polyols is preferably 1.8 to 3.2, particularly preferably 1.9 to 3.0.

Suitable polyether polyols are polyadducts of cyclic ethers with OH- or NH-functional starter molecules, said polyadducts optionally having a block structure.

Suitable cyclic ethers are, for example, styrene oxides, ethylene oxide, propylene oxide, tetrahydrofuran, butylene oxide, epichlorohydrin and any desired mixtures thereof.

Starters which may be used are the polyhydric alcohols mentioned in connection with the polyesterpolyols and having an OH functionality of ≧2 and primary or secondary amines and amino alcohols.

Preferred polyether polyols are those of the abovementioned type, exclusively based on propylene oxide or random or block copolymers based on propylene oxide with further 1-alkylene oxides, the proportion of 1-alkylene oxides being not higher than 80% by weight. Propylene oxide homopolymers and random or block copolymers which have oxyethylene, oxypropylene and/or oxybutylene units are particularly preferred, the proportion of the oxypropylene units, based on the total amount of all oxyethylene, oxypropylene and oxybutylene units, accounting for at least 20% by weight, preferably at least 45% by weight. Here, oxypropylene and oxybutylene comprise all respective linear and branched C₃- and C₄-isomers.

Such polyetherpolyols preferably have number average molar masses of 250 to 10 000 g/mol, particularly preferably of 500 to 8500 gimol and very particularly preferably of 600 to 4500 g/mol. The OH functionality is preferably 1.5 to 4.0, particularly preferably 1.8 to 3.1.

In addition, low molecular weight aliphatic, araliphatic or cycloaliphatic di-, tri- or polyfunctional alcohols having molecular weights below 500 g/mol, and being short-chain, i.e., containing 2 to 20 carbon atoms, are also useful as polyfunctional, isocyanate-reactive compounds as constituents of polyol component b).

These can be for example ethylene glycol, diethylene glycol, triethylene glycol, tetraethylene glycol, dipropylene tripropylene glycol, 1,2-propanediol, 1,3-propanediol, 1,4-butanediol, neopentyl glycol, 2-ethyl-2-butylpropanediol, trimethylpentanediol, positionally isomeric diethyloctanediols, 1,3-butylene glycol, cyclohexanediol, 1,4-cyclohexanedimethanol, 1,6-hexanediol, 1,2-cyclohexanediol, 1,4-cyclohexanediol, hydrogenated bisphenol A (2,2-bis(4-hydroxycyclohexyl)propane), 2,2-dimethyl-3-hydroxypropyl 2,2-dimethyl-3-hydroxypropionate. Examples of suitable triols are trimethylolethane, trimethylolpropane or glycerol. Suitable higher-functional alcohols are ditrimethylolpropane, pentaerythritol, dipentaerythritol or sorbitol.

It is also particularly preferable for the polyol component to be a difunctional polyether- or polyester or a polyether-polyester block copolyester or a polyether-polyester block copolymer having primary OH groups.

Particular preference is given to a combination of components a) and b) in the production of matrix polymers consisting of addition products of butyrolactone, e-caprolactone and/or methyl ε-caprolactone onto polyetherpolyols having a functionality of 1.8 to 3.1 with number average molar masses of 200 to 4000 g/mol in conjunction with isocyanurates, uretdiones, iminooxadiazinediones and/or other oligomers based on HDI. Very particular preference is given to addition products of ε-caprolactone onto poly(tetrahydrofurans) having a functionality of 1.9 to 2.2 and number average molar masses of 500 to 2000 g/mol (especially 600 to 1400 g/rnol), the number average overall molar mass of which is from 800 to 4500 g/mol and especially from 1000 to 3000 g/rnol, in conjunction with oligomers, isocyanurates and/or iminooxadiazinediones based on HDI.

The photoinitiators used are typically initiators which are activatable by actinic radiation and which trigger a polymerization of the corresponding polymerizable groups. Photoinitiators are commercially available compounds known per se, which are classed as unimolecular (type I) and bimolecular (type II). Type II photoinitiators may comprise in particular a cationic dye and a co-initiator. Useful co-initiators include ammonium arylborates as described for example in EP-A 0223587. Useful ammonium arylborates include, for example, tetrabutylammonium triphenylhexylborate, tetrabutylammonium triphenylbutylborate, tetrabutylammoniurn trinaphthylhexylborate, tetrabutylammonium tris(4-tert-butyl)phenylbutylborate, tetrabutylammonium tris(3-fluorophenyl)hexylborate, tetramethylammonium triphenylbenzylborate, tetra(n-hexyl)ammonium (sec-butyl)triphenylborate, 1-methyl-3-octylimidazolium dipentyldiphenylborate and tetrabutylammonium tris(3-chloro-4-methylphenyl)hexylborate (Cunningham et al., RadTech'98 North America UV/EB Conference Proceedings, Chicago, Apr. 19-22, 1998).

It can be advantageous to use mixtures of these compounds. Depending on the radiation source used for curing, photoinitiator type and concentration have to be conformed in a manner known to a person skilled in the art. Further particulars are described for example in P. K. T. Oldring (Ed.), Chemistry & Technology of UV & EB Formulations For Coatings, Inks & Paints, Vol. 3, 1991, SITA Technology, London, pp. 61-328.

Preferred photoinitiators are mixtures of tetrabutylammonium tetrahexylborate, tetrabutylammonium triphenylhexylborate, tetrabutylammonium tris(3-fluorophenyl)hexylborate ([191726-69-9], CGI 7460, product from BASF SE, Basle) and tetrabutylammonium tris(3-chloro-4-methylphenyl)hexylborate ([1147315-11-4], CGI 909, product from BASF SE, Basle) with the F+An- dyes of the present invention.

One further preferred embodiment provides that the photopolymer formulation further comprises urethanes as plasticizers, wherein the urethanes may be more particularly substituted with at least one fluorine atom.

The urethanes may preferably be of general formula (I)

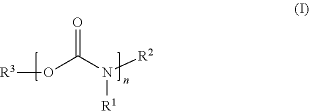

where n is ≧1 and ≦8 and R³ is a linear, branched, cyclic or heterocyclic unsubstituted or else optionally heteroatom-substituted organic moiety and/or R² and R³ are each independently hydrogen, wherein preferably at least one of R¹, R² and R³ is substituted with at least one fluorine atom and more preferably R³ is an organic moiety having at least one fluorine atom. It is particularly preferably for R¹ to be a linear, branched, cyclic or heterocyclic organic moiety which is unsubstituted or else optionally substituted with heteroatoms such as fluorine for example.

A further preferred embodiment provides that the writing monomer comprises at least one mono-and/or multifunctional writing monomer, wherein mono- and multifunctional acrylate writing monomers may be concerned in particular. It may be particularly preferable for the writing monomer to comprise at least one monofunctional and one multifunctional urethane(meth)acrylate.

Acrylate writing monomers may concern in particular compounds of general formula (II)

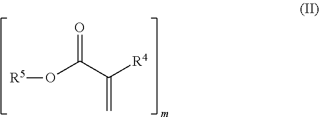

in each of which m is ≧1 and ≦4 and R⁵ is a linear, branched, cyclic or heterocyclic unsubstituted or else optionally heteroatom-substituted organic moiety and/or R⁴ is hydrogen, a linear, branched, cyclic or heterocyclic unsubstituted or else heteroatom-substituted organic moiety. It is particularly preferable for R⁴ to be hydrogen or methyl and/or R⁵ to be a linear, branched, cyclic or heterocyclic unsubstituted or else optionally heteroatom-substituted organic moiety.

It is similarly possible for further unsaturated compounds such as αβ-unsaturated carboxylic acid derivatives such as acrylates, methacrylates, maleates, fumarates, maleimides, acrylamides, also vinyl ethers, propenyl ethers, allyl ethers and dicyclopentadienyl-containing compounds and also olefinically unsaturated compounds such as, for example, styrene, α-methylstyrene, vinyltoluene, olefins, e.g., 1-octene and/or 1-decene, vinyl esters, (meth)acrylonitrile, (meth)acrylamide, methacrylic acid, acrylic acid to be added. Acrylates and methacrylates are preferable, however.

Esters of acrylic acid and of methacrylic acid are generally referred to as acrylates and methacrylates, respectively. Examples of usable acrylates and methacrylates are methyl acrylate, methyl methacrylate, ethyl acrylate, ethyl methacrylate, ethoxyethyl acrylate, ethoxyethyl methacrylate, n-butyl acrylate, n-butyl methacrylate, tert-butyl acrylate, tert-butyl methacrylate, hexyl acrylate, hexyl methacrylate, 2-ethylhexyl acrylate, 2-ethylhexyl methacrylate, butoxyethyl acrylate, butoxyethyl methacrylate, lauryl acrylate, lauryl methacrylate, isobornyl acrylate, isobornyl methacrylate, phenyl acrylate, phenyl methacrylate, p-chlorophenyl acrylate, p-chlorophenyl methacrylate, p-bromophenyl acrylate, p-brotnophenyl methacrylate, 2,4,6-trichlorophenyl actylate, 2,4,6-trichlorophenyl methacrylate, tribromophenyl acrylate, 2,4,6-tribromophenyl methacrylate, pentachlorophenyl acrylate, pentachlorophenyi methacrylate, pentabromophenyl acrylate, pentabromophenyl methacrylate, pentabromobenzyl acrylate, pentabromobenzyl methacrylate, phenoxyethyl acrylate, phenoxyethyl methacrylate, phenoxyethoxyethyl acrylate, phenoxyethoxyethyl methacrylate, phenyltliioethyl acrylate, phenylthioethyl methacrylate, 2-naphthyl acrylate, 2-naphthyl methacrylate, 1,4-bis(2-thionaphthyl)-2-butyl acrylate, 1,4-bis(2-thionaphthyl)-2-butyl methacrylate, propane-2,2-diylbis[(2,6-dibromo-4,1-phenylene)oxy(2-{[3,3,3-tris(4-chlorophenyl)propanoyl]oxy}propane-3,1-diypoxyethane-2,1-diyl ]diacrylate, bisphenol A diacrylate, bisphenol A dimethacrylate, tetrabromohisphenol A diacrylate, tetrabromobisphenol A dimethacrylate and also the ethoxylated analog compounds thereof, N-carbazolyl acrylates, to mention but a selection of usable acrylates and methacrylates.

It will be appreciated that further urethane acrylates may also be used. Urethane acrylates are compounds having at least one acrylic ester group and in addition at least one urethane bond. It is known for compounds of this type to be obtainable by reacting a hydroxyl-functional acrylic ester with an isocyanate-functional compound.

Examples of isocyanate-functional compounds usable for this include aromatic, araliphatic, aliphatic and cycloaliphatic di-, tri- or polyisocyanates. Mixtures of such di-, tri- or polyisocyanates are also usable. Examples of suitable di-, tri- or polyisocyanates include butylene diisocyanate, hexamethylene diisocyanate (HDI), isophorone diisocyanate (IPDI), 1,8-diisocyanato-4-(isocyanatomethyl)octarie, 2,2,4- and/or 2,4,4-trimethylhexamethylerte diisocyanate, the isomeric bis(4,4′-isocyanatocyclohexyl)methanes and their mixtures of any desired isomeric content, isocyanatomethyl-1,8-octane diisocyanate, 1,4-cyclohexylene diisocyanate, the isomeric cyclohexanedimethylene diisocyanates, 1,4-phenylene diisocyanate, 2,4- and/or 2,6-tolylene diisocyanate, 1,5-naphthylene diisocyanate, 2,4′- or 4,4′-diphenylmethane diisocyanate, 1,5-naphthylene diisocyanate, m-methylthiophenyl isocyanate, triphenylmethane 4,4′, 4″-triisocyanate and tris(p-isocyanatophenyl)thiophosphate or their urethane-, urea-, carbodiimide-, acylurea-, isocyanurate-, allophanate-, biuret-, oxadiazinetrione-, uretdione- or iminooxadiazinedione-structured derivatives and mixtures thereof. Aromatic or araliphatic di-, tri- or polyisocyanates are preferable here.

Useful hydroxyl-functional acrylates or methacrylates for preparing urethane acrylates include, for example, compounds such as 2-hydroxyethyl(meth)acrylate, polyethylene oxide mono(meth)acrylates, polypropylene oxide mono(meth)acrylates, polyalkylene oxide mono(meth)acrylates, poly(ε-caprolactone) mono(meth)acrylates, e.g., Tone® M100 (Dow, Schwalbach, DE), 2-hydroxypropyl(meth)acrylate, 4-hydroxybutyl(meth)acrylate, 3-hydroxy-2,2-dimethylpropyl(meth)acrylate, hydroxypropyl(meth)acrylate, 2-hydroxy-3-phenoxypropyl acrylate, the hydroxyl-functional mono-, di- or tetraacrylates of polyhydric alcohols such as trimethylolpropane, glycerol, pentaerythritol, dipentaerythritol, ethoxylated, propoxylated or alkoxylated trimethy iolpropane, glycerol, pentaerythritol, dipentaerythritol or technical-grade mixtures thereof. Preference is given to 2-hydroxyethyl acrylate, hydroxypropyl acrylate, 4-hydroxybutyl acrylate and poly(ε-caprolactone) mono(meth)acrylates. Also suitable are isocyanate-reactive oligomeric or polymeric unsaturated acrylate and/or methacrylate compounds alone or in combination with the aforementioned monomeric compounds. It is likewise possible to use the known hydroxyl-containing epoxy(meth)acrylates having OH contents of 20 to 300 mg KOH/g or hydroxyl-containing polyurethane(meth)acrylates having OH contents of 20 to 300 mg KOH/g or acrylated polyacrylates having OH contents of 20 to 300 mg KOH/g and also their mixtures with each other and mixtures with hydroxyl-containing unsaturated polyesters and also mixtures with polyester(meth)acrylates or mixtures of hydroxyl-containing unsaturated polyesters with polyester(meth)acrylates.

The present invention further provides compounds of the formulae

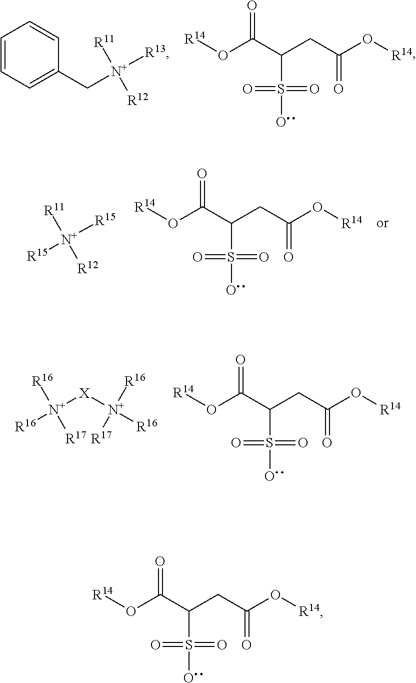

where
R¹¹ and R¹² are each independently methyl, ethyl, propyl, butyl, hydroxyethyl or cyanoethyl,
R¹³ is C₁₆- to C₂₂-alkyl or is C₁₀- to C₂₂-alkyl when R¹ and R² are not both methyl.
R¹⁴ is optionally branched C₆- to C₁₂ alkyl,
R ¹⁵ is C₁₂- to C₂₂-alkyl,
R¹⁶ and R¹⁷ are each independently methyl, ethyl, propyl or butyl,
R¹⁷ is additionally benzyl,
X is a —(CH₂)ₙ— bridge, and
n is an integer from 4 to 10.
These compounds are ammonium salts. It is these compounds in particular which are useful as liquid ink or liquid ink constituent in the method of the present invention, although the use of these compounds is explicitly not restricted thereto. These compound of the present invention are preferably characterized in that
R¹¹ and R each independently methyl, ethyl or hydroxyethyl, in particular methyl,
R¹³ is hexadecyl or octadecyl,
R¹⁴ is n-hexyl, n-octyl, 2-ethylhexyl or decyl, in particular 2-ethylhexyl,
R¹⁵ is dodecyl, tetradecyl, hexadecyl or octadecyl, in particular hexadecyl or octadecyl,

R¹⁶ and R¹⁷ are each independently methyl, ethyl, propyl or butyl, in particular propyl or butyl,
X is a —(CH₂)ₙ— bridge, and
n is an integer from 4 to 8, in particular 6.

The chemical makeup of component 1 of the liquid printing ink according to the present invention will now be described.

The active substances of component 1 are substances which absorb in the UV region, the visible region and/or the IR region of the electromagnetic spectrum.

Substances which absorb in the UV region are organic substances without extended π-system and also UV absorbers and whiteners. Also included are rare earth complexes with fluorescence in the visible region.

Substances which absorb in the visible region are organic dyes.

Substances which absorb in the infrared (IR) region are organic IR dyes.

The substances concerned in all these cases dissolve in component 2 of the liquid printing ink according to the present invention, or their mixtures do.

Preference is given to substances having a glass transition temperature <20° C. Substances having a melting point<20° C. are likewise suitable.

The substances may be ionic or nonionic compounds.

UV absorbers or neutral whiteners are examples of nonionic substances which absorb in the UV region.

Examples of ionic substances absorbing in the UV region include, for example, alkali metal, ammonium, sulfonium, phosphonium or cycloimmonium salts of colorless anions, as well as cationic or anionic whiteners.

Neutral dyes are examples of nonionic substances absorbing in the visible region.

Cationic or anionic dyes are examples of ionic substances absorbing in the visible region.

Neutral IR dyes are examples of nonionic substances absorbing in the IR region.

Cationic or anionic IR dyes are examples of ionic substances absorbing in the IR region.

Whiteners, dyes and IR dyes are known fbr example from H. Zollinger, Color Chemistry, Wiley-VCH, 3rd edition, 2003. UV absorbers are known for example from J. Bieleman, Lackadditive, Wiley-VCH, 1998, chapter 8.2.

Ionic compounds are preferable.

The molar mass is preferably above 200 but below 1000.

Alkali metal, ammonium, sulfoniurn, phosphonium, cycloammonium or cycloimmonium ions are:

Lithium, sodium, potassium;

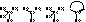

where

R²¹ to R²⁵ are each independently optionally substituted C₁- to C₂₂-alkyl, C₃- to C₅-cycloalkyl or C₇ to C₁₀-aralkyl moieties and

R²¹ may additionally be optionally substituted phenyl.

Colorless anions are: C₈- to C₂₅-alkanesulfonate, preferably C₁₃- to C₂₅-alkanesulfonate, C₃ to C₁₈-perfluoroalkanesulfonate, preferably C₄ to C₁₈-perfluoroalkanesulfonate, C₈- to C₂₅-alkanoate, C₉- to C₂₅-alkenoate, C₈- to C₂₅-alkylsulfate, preferably C₁₃- to C₂₅-alkylsulfate, C₈- to C₂₅-alkenylsulfate, preferably C₁₃- to C₂₅-alkenylsulfate, C₃- to C₁₈-perfluoroalkylsulfate, preferably C₄- to C₁₈-perfluoroalkylsulfate, polyether sulfates based on at least 4 equivalents of ethylene oxide and/or equivalents 4 of propylene oxide, bis-C₄- to C₂₅-alkyl-, C₅- to C₇-cycloalkyl-, C₃- to C₈-alkenyl- or C₇- to C₁₁-aralkyl-sulfosuccinate, bis-C₂- to C₁₀-alkyisulfosuccinate substituted by at least 8 fluorine atoms, C₈- to C₂₅-alkylsulfoacetates, benzenesulfonate substituted by at least one moiety from the group halogen, C₄- to C₂₅-alkyl, perfluoro-C₁- to C₈-alkyl and/or C₁- to C₁₂-alkoxycarbonyl, naphthalene- or biphenylsulfonate, optionally substituted by nitro, cyano, hydroxyl, C₁- to C₂₅-alkyl, C₄- to C₁₂-alkoxy, amino, C₁- to C₁₂-alkoxycarbonyl or chlorine, benzene-, naphthalene- or biphenyidisulfate optionally substituted by nitro, cyano, hydroxyl, C₁- to C₂₅-alkyl, C₁- to C₁₂-alkoxy, C₁- to C₁₂-alkoxycarbonyl or chlorine, benzoate substituted by dinitro, C₆- to C₂₅-alkyl, C₄- to C₁₂-alkoxycarbortyl, benzoyl, chlorobenzoyl toluoyl, the anion of naphthalenedicarboxylic acid, diphenyl ether disulfonate, sulfonated or sulfated, optionally at least monounsaturated C₈- to C₂₅-fatty acid esters of aliphatic C₁- to C₈-alcohols or glycerol, bis(sulfo-C₂- to C₆-alkyl) C₃- to C₁₂-alkanedicarboxylates, bis(sulfo-C₂- to C₆-alkyl)itaconates, (sulfo-C₂- to C₆-alkyl) C₆- to C₁₈-alkanecarboxylates, (sulfo-C₂- to C₆-alkyl)acrylates or methacrylates, triscatechol phosphate optionally substituted by up to 12 halogen moieties, an anion from the group tetraphenylborate, cyanotriphenylborate, tetraphenoxyborate, C₄- to C₁₂-alkyltriphenylborate whose phenyl or phenoxy moieties may be substituted by halogen, C₁- to C₄-alkyl and/or C₁- to C₄-alkoxy, C₄- to C₁₂-alkyltrinaphthylborate, tetra-C₁- to C₂₀-alkoxyborate, 7,8- or 7,9-dicarbanidoundecaborate(1-) or (2-), which are optionally substituted on the boron and/or carbon atoms by one or two C₁- to C₁₂-alkyl or phenyl groups, dodecahydrodicarbadodecaborate(2-) B-C₁- C₁₂alkyl-C-phenyidodecahydrodicarbadodecaborate(1-), wherein in the case of polyvalent anions such as naphthalenedistilfonate, An- represents one equivalent of this anion, and wherein the alkane and alkyl groups may be branched and/or may be substituted by halogen, cyano, methoxy, ethoxy, methoxycarbonyl or ethoxycarbonyl.

Particular preference is given to:

sec-C₁₁- to C₁₈-alkanesulfonate, C₁₃- to C₂₅-alkylstilfate, branched C₈- to C₂₅-alkylsulfate, optionally branched bis-C₆- to C₂₅-alkylsulfosuccinate, sec- or tert-C₄- to C₂₅-alkylbenzenesulfonate, sulfonated or sulfated, optionally at least monounsaturated C₈- to C₂₅-fatty acid esters of aliphatic C₁- to C₈-alcohols or glycerol, bis-(sulfo-C₂- to C₆-alkyl) C₃- to C₁₂-alkanedicarboxylates, (sulfo-C₂- to C₆-alkyl) C₆- to C₁₈-alkanecarboxylates, triscatechol phosphate substituted by up to 12 halogen moieties, cyanotriphenylborate, tetraphenoxyborate.

Examples are:

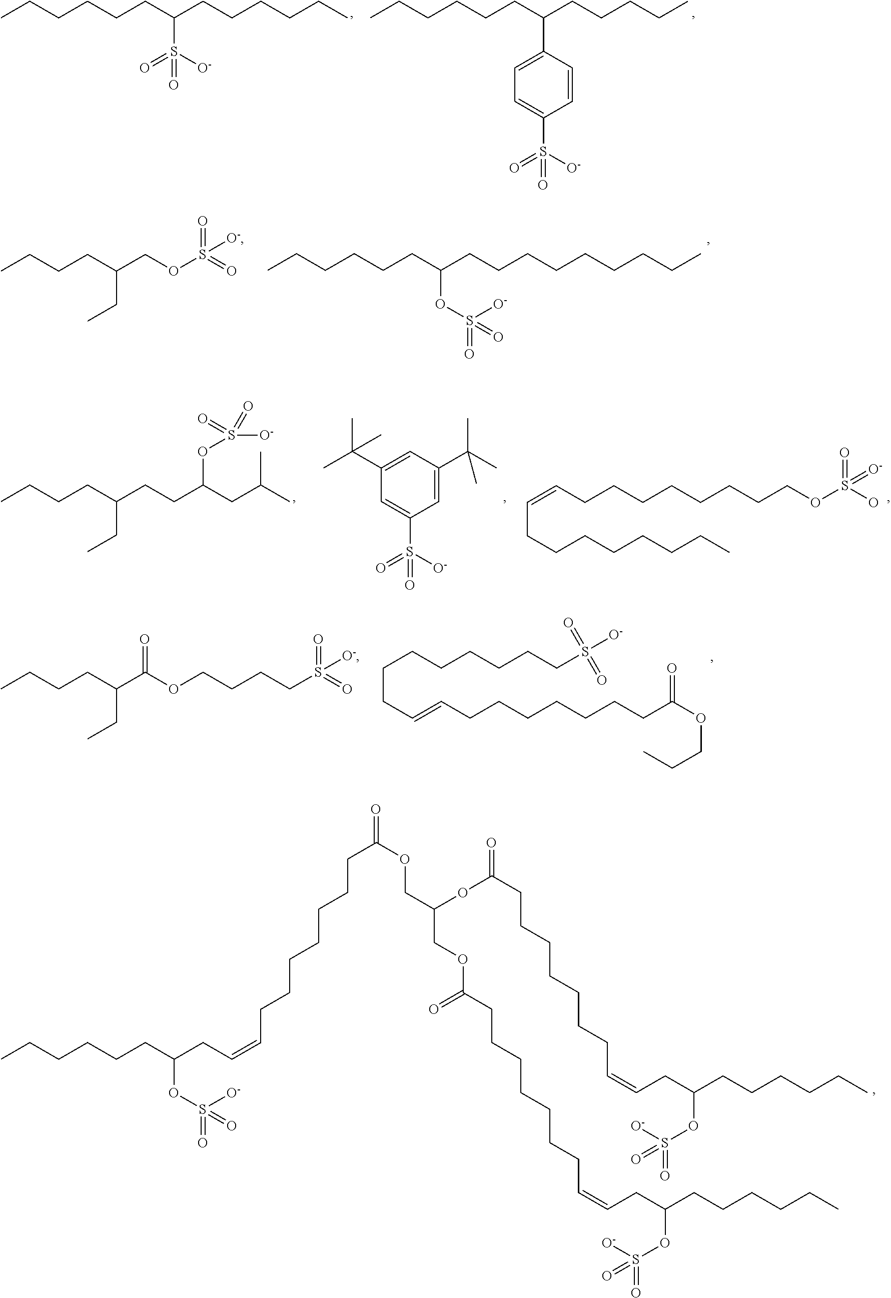

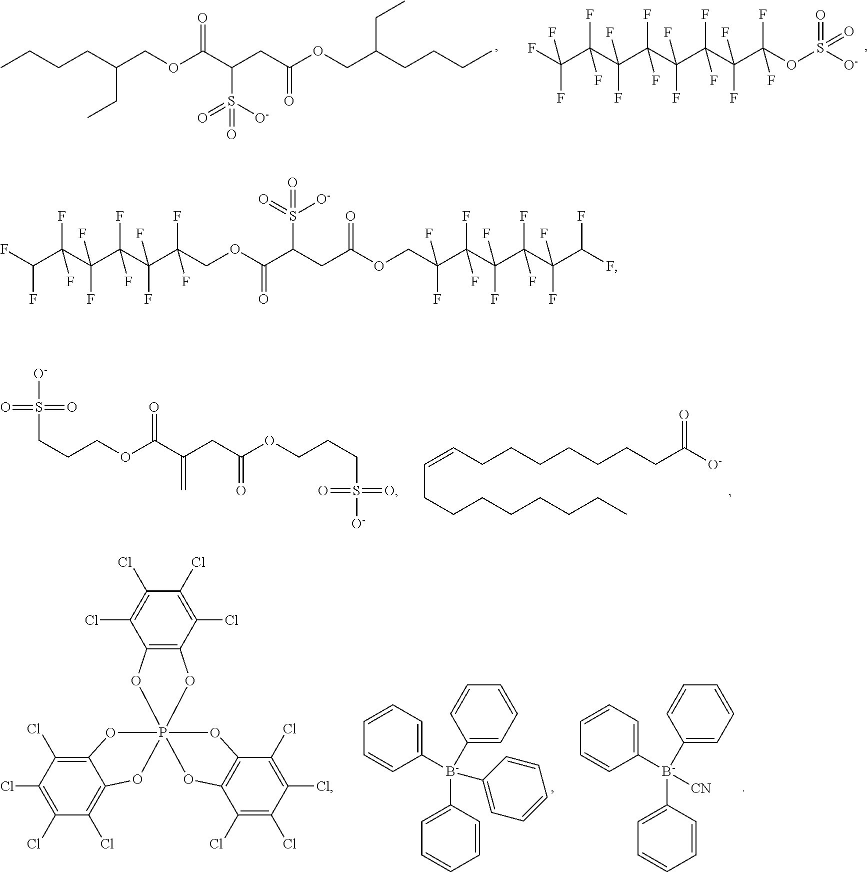

Preferred colorless salts are ionic liquids of the type which is commercially available. Likewise preferred colorless salts are ammonium, sulfonium, phosphonium, cycloammonium or cycloimmonium salts of organic mono-, bis- or trissulfonic acids, wherein not only the cation but also the anion each bear at least one long-chain, optionally branched alkyl moiety, Long-chain alkyl moieties are those having at least 6, preferably at least 8, more preferably at least 10, still more preferably at least 12 and most preferably at least 16 carbon atoms. It is likewise to be understood as meaning that the overall number of carbon atoms is at least 12, preferably at least 18 and more preferably at least 24 when the cation or anion bears at least two alkyl groups.

Examples of colorless salts are:

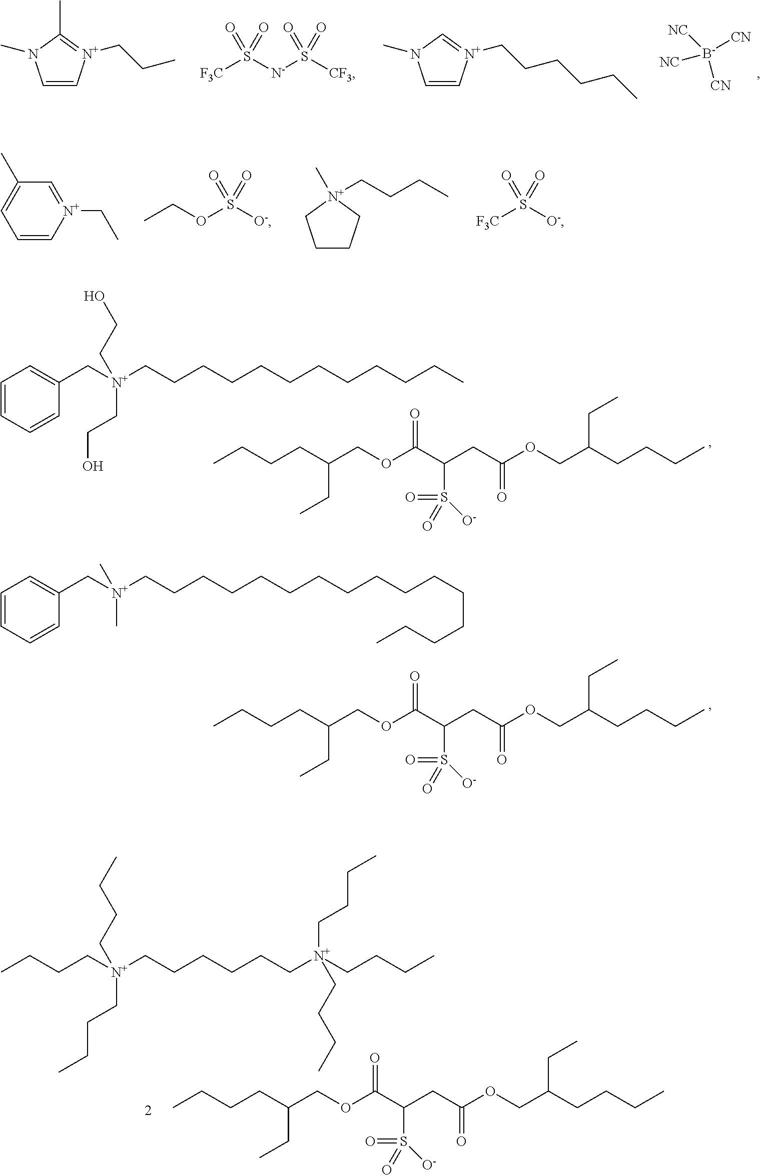

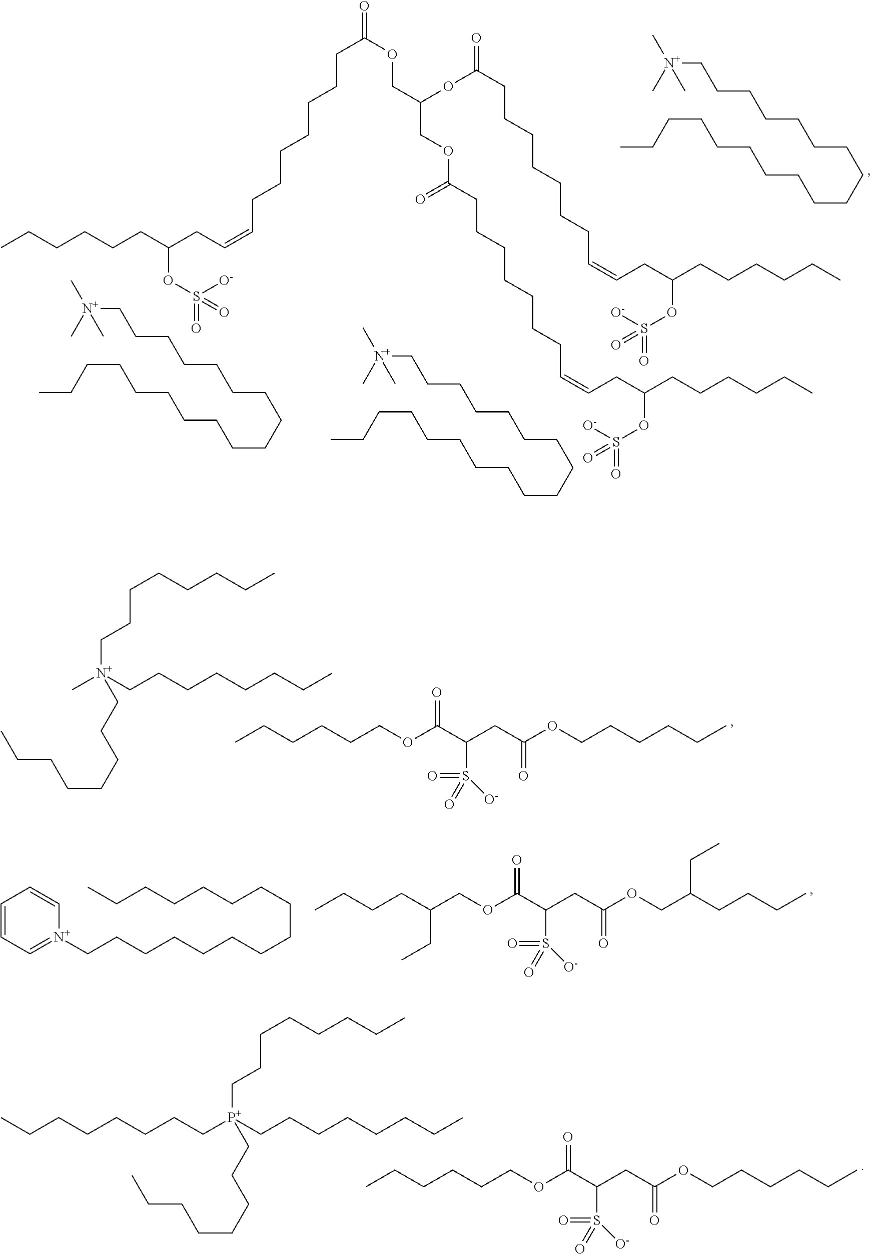

Examples of whiteners are:

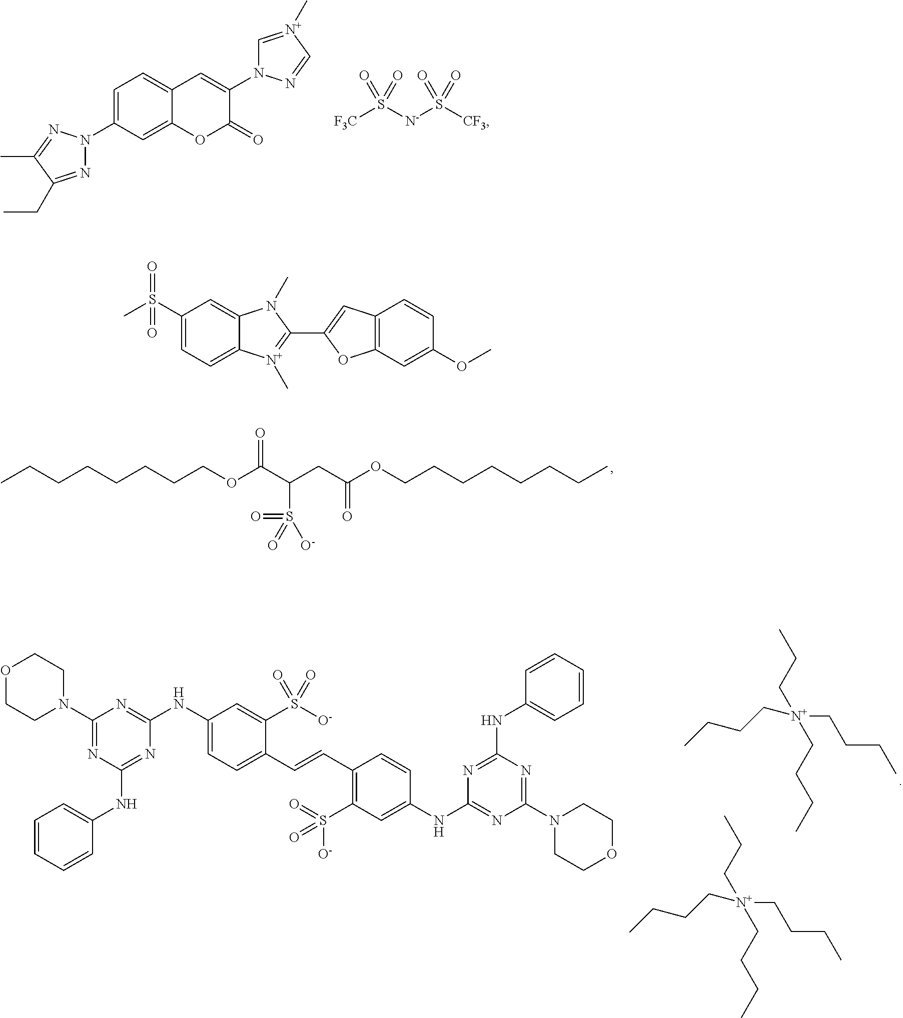

Examples of rare earth complexes are preferably those of europium, of therbiurn, of thul and of dysprosium, e.g.:

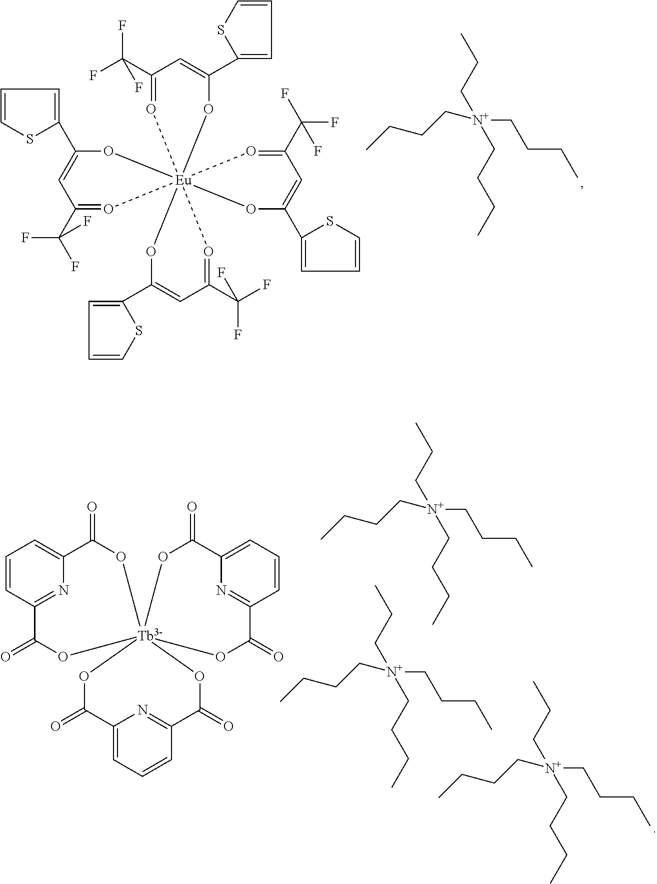

Preferred ionic dyes are cationic dyes of the type known for example from H. Berneth in Ullmann's Encyclopedia of industrial Chemistry, Cationic Dyes, Wiley-VCH Verlag, 2008. They preferably belong to the following classes: acridine dyes, xanthene dyes, thioxanthene dyes, phenazine dyes, phenoxazine dyes, phenothiazine dyes, coumarin dyes, tri(het)arylmethane dyes, in particular diamino-and triamino(het)arylmethane dyes, mono-, di- and trimethinecyanine dyes, hemicyanine dyes, diazahemicyanine dyes, zeromethine dyes, in particular naphtholactam dyes, streptocyanine dyes, externally cationic merocyanine dyes, externally cationic neutrocyanine dyes, externally cationic phthalocyanine dyes, externally cationic anthraquinone dyes, externally cationic azo dyes. Such dyes are described for example in H. Berneth in Ullmann's Encyclopedia of Industrial Chemistry, Azine Dyes, Wiley-VCH Verlag, 2008, H. Berneth in Ullmann's Encyclopedia of Industrial Chemistry, Methine Dyes and Pigments, Wiley-VCH Verlag, 2008, T. Gessner, U. Mayer in Ullmann's Encyclopedia of Industrial Chemistry, Triarylrnethane and Diarylmethane Dyes, Wiley-VCH Verlag, 2000, H.-S. Bien, J. Stawitz, K. Wunderlich in Ullmann's Encyclopedia of Industrial Chemistry, Anthraquinone Dyes and Intermediates, Wiley-VCH Verlag, 2008, K. Hunger, P.Mischke, W. Rieper, R. Raue, K. Kunde, A. Engel in Ullmann's Encyclopedia of Industrial Chemistry, Azo Dyes, Wiley-VCH Verlag, 2008.

Useful anions include any colorless anions but also colored anions, for example chloride, nitrate, phosphate, sulfate, acetate, PF₆, perchlorate, methosulfate, methanesulfonate, tritluoromethanesulfonate, toluenesulfonate, tetraphenylborate, anionic dyes, anions of organic mono-, bis- or trissulfonic acids which each bear at least one long-chain, optionally branched alkyl moiety. Long-chain alkyl moieties are those having at least 8, preferably at least 10, more preferably at least 12, still more preferably at least 14 and most preferably at least 16 carbon atoms. It is likewise to be understood as meaning that the overall number of carbon atoms is at least 12, preferably at least 18 and more preferably at least 24 when the anion bears at least two alkyl groups.

Preferred anions are those mentioned last.

Examples of cationic dyes are:

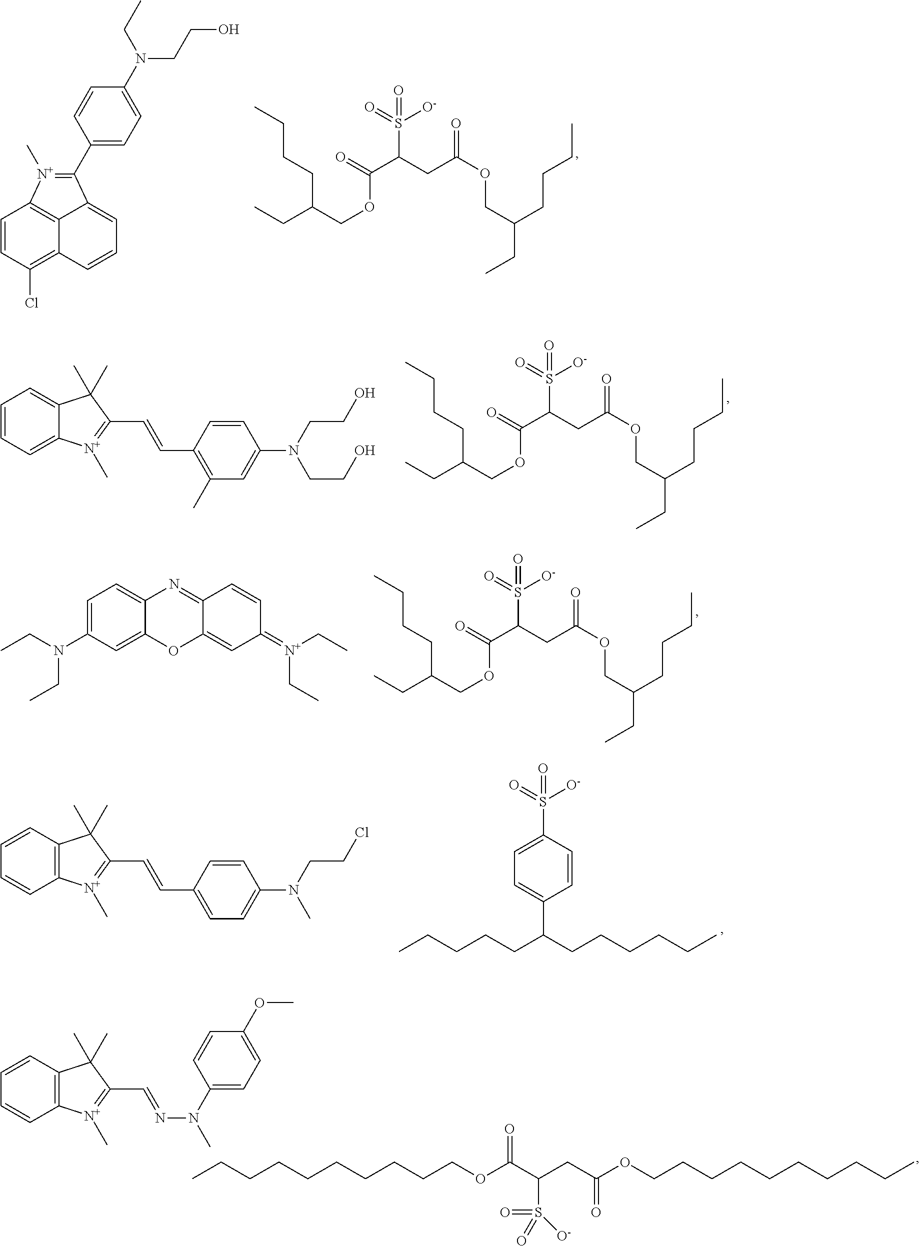

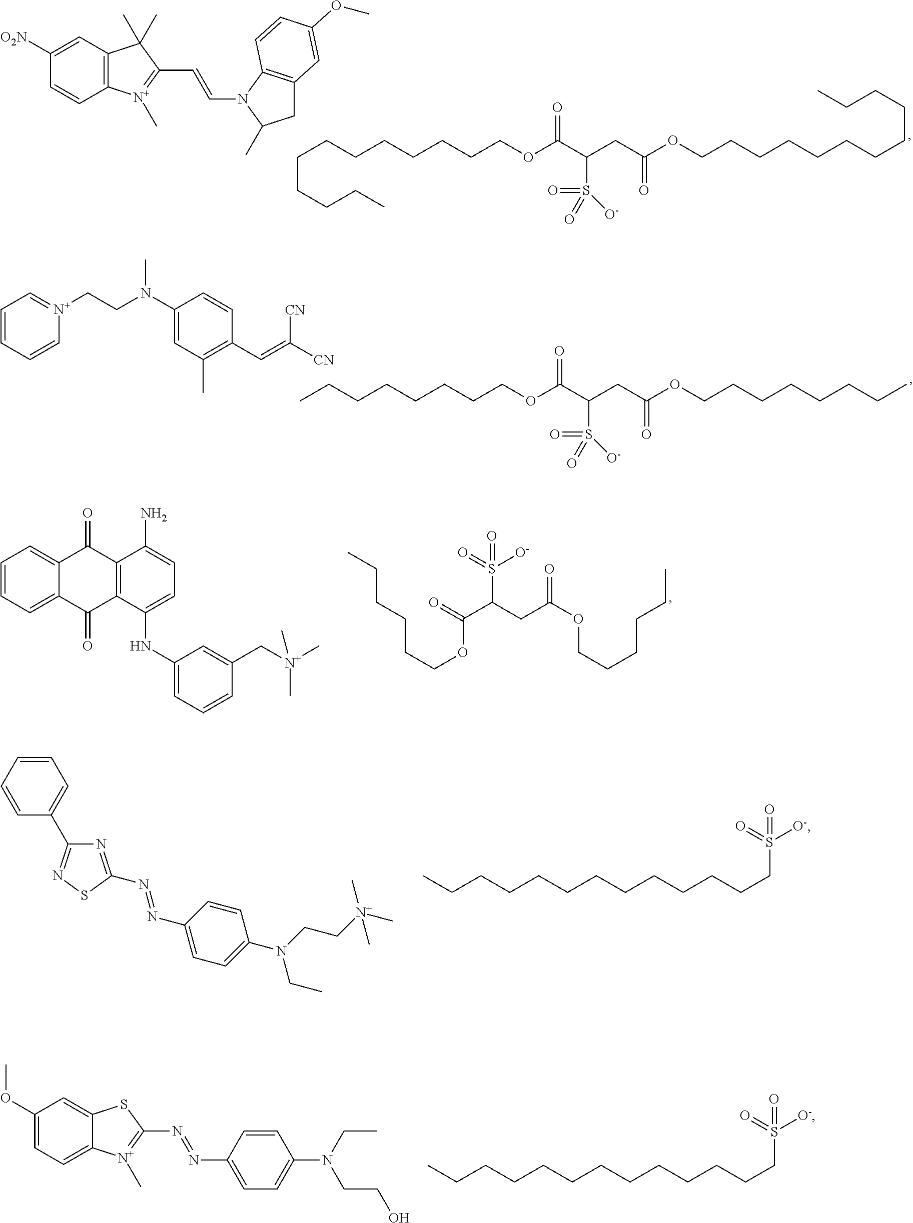

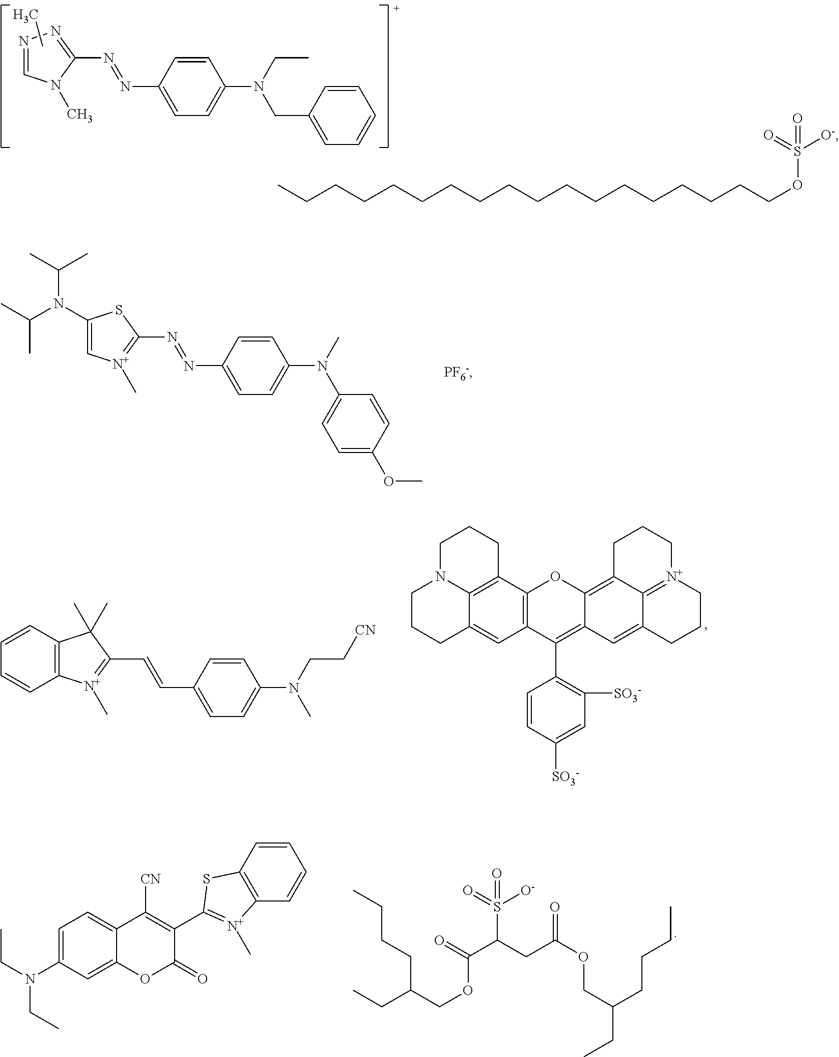

Anionic dyes are likewise preferred ionic dyes. They preferably belong to the following classes: oxonols, di- and trihydroxytriarylmethane dyes, the group of merocyanine, neutrocyanine, coumarin, anthraquinone, anthrapyridone, dioxazine, mono-, dis- and trisazo dyes having at least one sulfo group, the group of acridine, xanthene, thioxanthene, phenazine, phenoxazine, phenothiazine, tri(het)arylmethane dyes, in particular diamino- and triamino(het)arylmethane dyes, having at least two sulfo groups. Such dyes are described for example in H. Berneth in Ullmann's Encyclopedia of Industrial Chemistry, Azine Dyes, Wiley-VCH Verlag, 2008, H. Berneth in Ullmann's Encyclopedia of industrial Chemistry, Methine Dyes and Pigments, Wiley-VCH Verlag, 2008, T. Gessner, U. Mayer in Ullmann's Encyclopedia of industrial Chemistry, Triarylmethane and Diarylmethane Dyes, Wiley-VCH Verlag, 2000, H.S. Bien, J. Stawitz, K. Wunderlich in Ullmann's Encyclopedia of Industrial Chemistry, Anthraquinone Dyes and Intermediates, Wiley-VCH Verlag, 2008, K. Hunger, P.Mischke, W. Rieper, R. Raue, K. Kunde, A. Engel in Ullmann's Encyclopedia of Industrial Chemistry, Azo Dyes, Wiley-VCH Verlag, 2008. Likewise preferred anionic dyes are phthalocyanines and azo metal complexes bearing at least one sulfo group. Such dyes are described for example in Gert Lobbert in Ullmann's Encyclopedia of Industrial Chemistry, Phthalocyanines, Wiley-VCH Verlag, 2000 and Klaus Grychtol, Winfried Mennicke in Ullmann's Encyclopedia of industrial Chemistry, Metal-Complex Dyes, Wiley-VCH Verlag, 2000.

Possible cations for use in such anionic dyes include the above-described alkali metal, ammonium, sulfonium, phosphonium or cycloimmonium ions. Tetralkylammonium and cycloimmonium ions are preferable.

Examples of anionic dyes are:

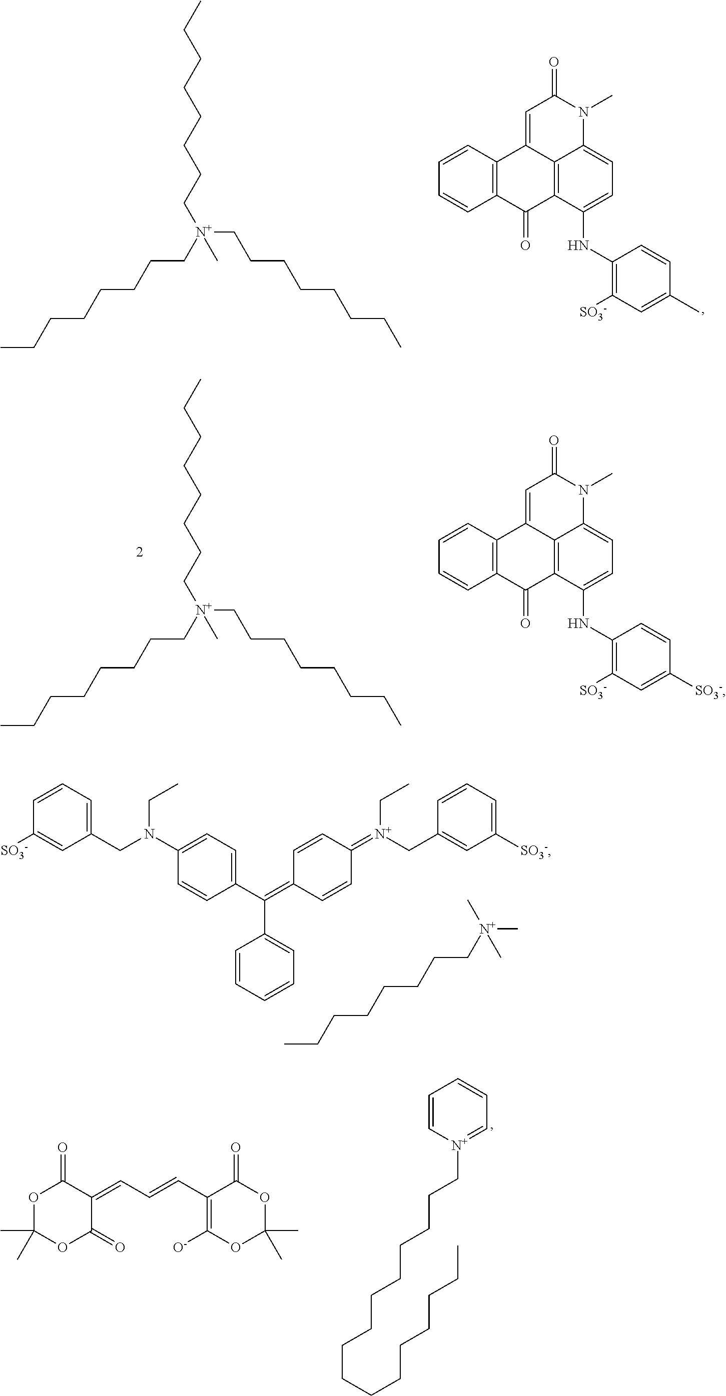

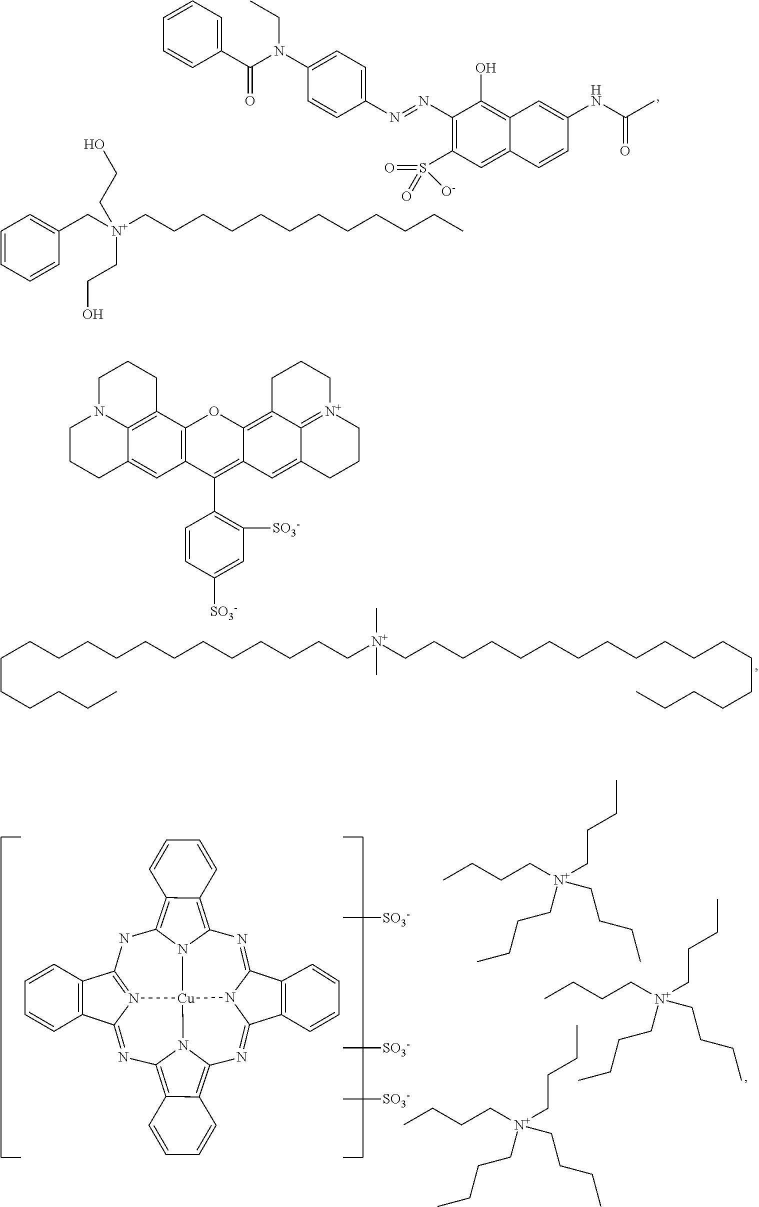

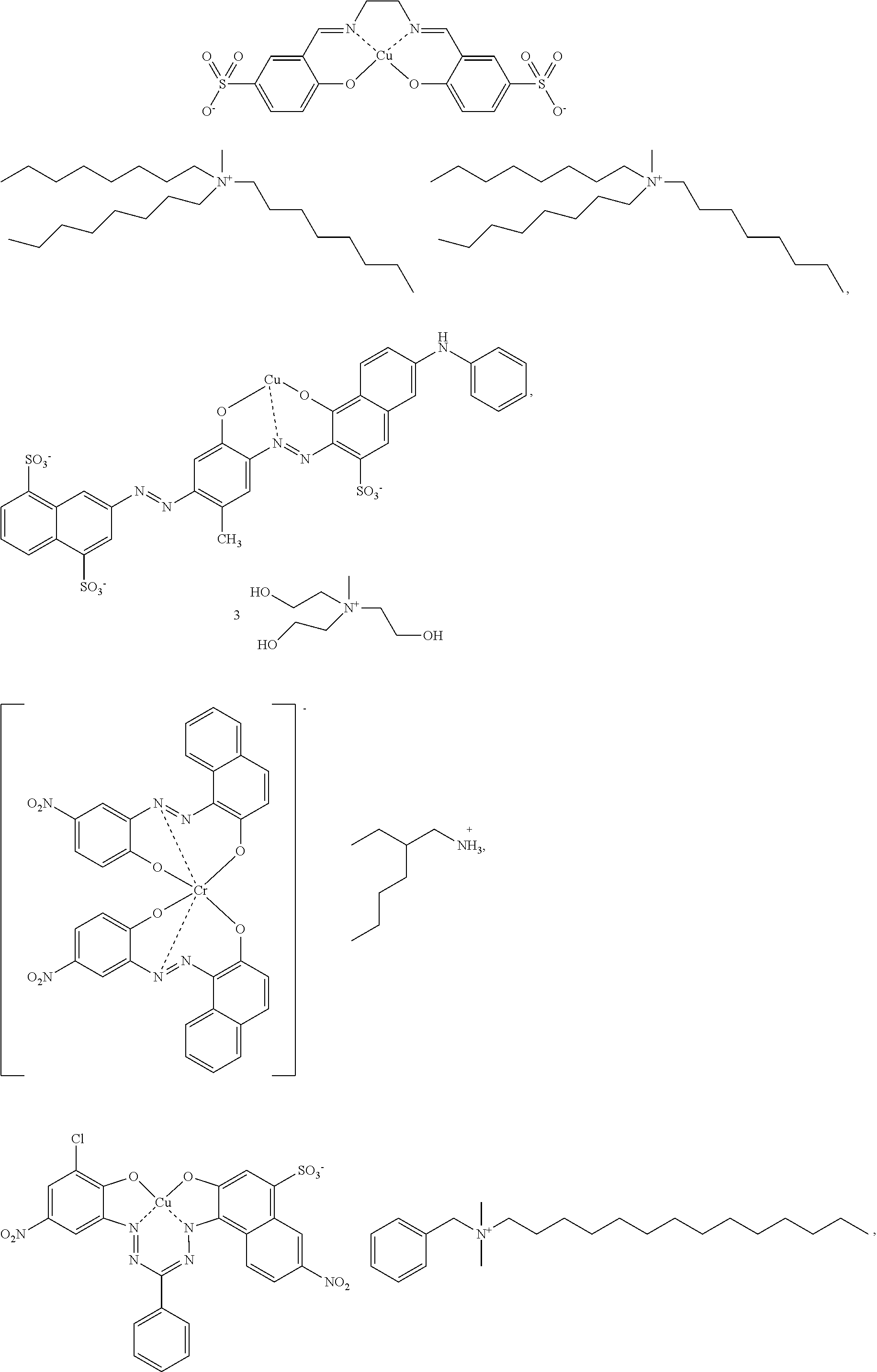

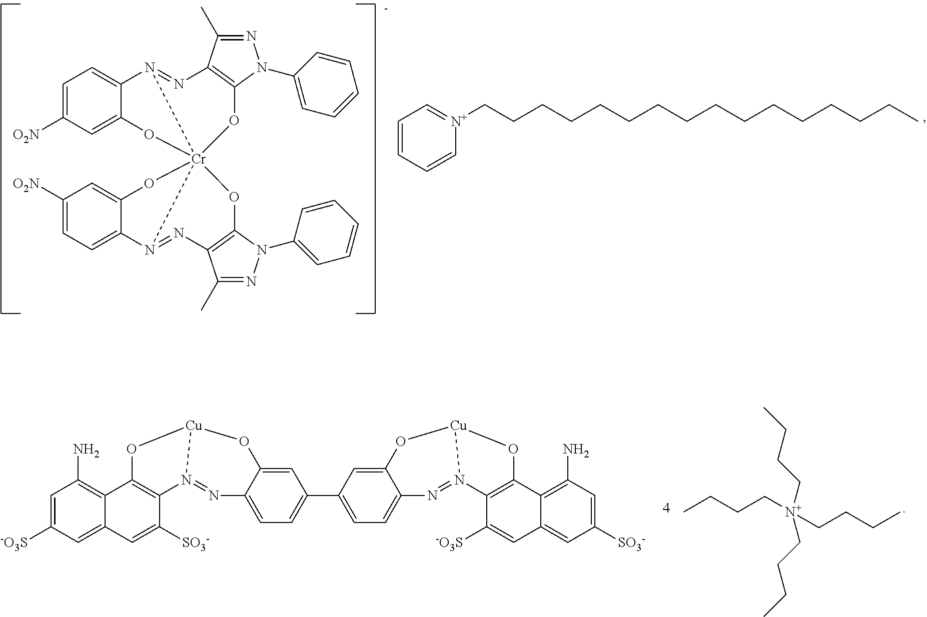

Suitable nonionic dyes include for example:

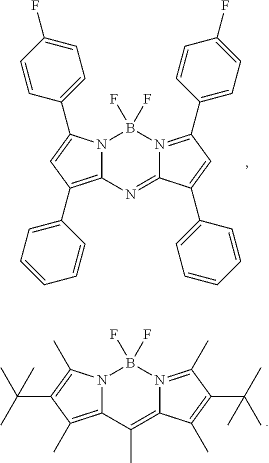

Suitable nonionic rare earth complexes are preferably those of europium, of therbium, of thulium and of dysprosium, e.g.:

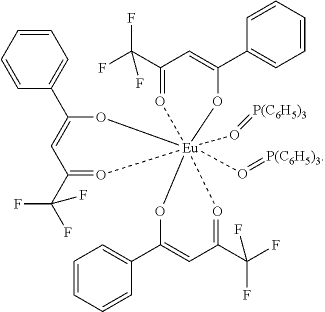

It is also possible for two or more of the abovementioned components 1 to be mixed, for example two or more dyes, two or more colorless salts or one or more colorless salts and one or more dyes. Preference is given to a mixture of a colorless salt and a dye, to a mixture of a colorless salt and a rare earth complex or to a mixture of a rare earth complex and a dye.

The chemical makeup of component 2 of the liquid printing ink according to the present invention will now be described.

Component 2 is a solvent having a boiling point between 60° C. and 240° C., preferably between 77° C. and 220° C. (all at 1013 mbar). It shall be capable of dissolving component 1. Mixtures of such solvents are likewise useful as component 2.

Useful solvents include, for example, 2-butanone, cyclohexanone, ethyl acetate, butyl acetate, methoxypropyl acetate or diethylene glycol monoethyl ether acetate.

The holographic process of exposure will now be described.

A multicolored volume reflection hologram is recorded using a copying film, a master (which carries the hologram to be copied) and two or more lasers of differing wavelengths. The copying film is based on the photopolymer whose light sensitivity matches the laser wavelengths, so the photopolymer will develop volume phase gratings on exposure to the laser or lasers. The master is a multicolored volume reflection hologram which reconstructs the hologram at the laser wavelengths used. Alternatively, the master is a digital element, for example a spatial light modulator (SLM). Alternatively, the master may also be a combination of volume reflection hologram and digital element. Industrial lasers of sufficient coherence, frequency stability and power output for holography are known, examples being frequency-doubled neodymium:YAG lasers, krypton ion lasers, argon ion lasers, helium-neon lasers and diode-pumped solid-state lasers.

Replication is the method commonly used for mass production of holograms, and it is based on the principle of contact exposure. This principle requires the photopolymer to be in contact with or close to the master hologram, for example at a distance of 0.2-2.0 cm, preferably 0.5-1.0 cm, more preferably about 1 cm, during the exposure phase. Typically, the master is either in the form of a plate or drum mounted as an arcuate sheetlike element. The photopolymer is a film which is on a substrate and which is laminated onto the master. Where the photopolymer has two substrates, for example a carrier and a concealer, the concealer is preferably removed before lamination to the master.

As noted, the color space is determined by the number of lasers. Mixed colors can be produced with two laser wavelengths, for example red-blue, blue-green or green-blue. Full-colored holograms include at least three lasers of sufficiently differing wavelengths, for example red-green-blue (ROB), to ensure good coverage of the color space. Usage of more than three lasers for hologram production is possible. We shall nonetheless proceed from the RGB scenario, since the principle described is applicable to the other scenarios.

In a first step, the RGB laser beams are diverted to create a white bundle of laser beams which is directed in a divergent manner and under a defined angle, for example close to 45°, onto a conformed reflection master hologram with matching reference angle. The master possesses the RGB spectral components, and the diffraction efficiency is individually conformed for every color, so the part beam ratio—intensity ratio of object beam to reference beam—which is ideal for the geometry and the copying film results. The laser beam may be set up to be sheetlike, or scanning as a line or as a sensing point beam. Preference is given to the line scan at a constant speed across the entire area of the master.

The stipulation in relation to laser exposure is the production of holograms with optimized brightness in all three colored components. A compromise has to be arrived at between the basic brightness of the hologram and the attainable chromaticities for the additive mixing colors in the form of an ideal setting for the RGB exposure conditions. Optimizing the exposure sequence (RGB order, exposure time per color, intensity per color, introduced amount of energy, overall intensity) gives bright colors and bright holograms and facilitates the ease of recognition and enhances the anti-counterfeit protection. The RGB exposure may be effected concurrently for all colors or with overlapping RGB sequence in the best color order for the photopolymer, or sequentially, i.e., in individual exposures, again in the order of colors which is best for the photopolymer.

During replication, the so-called reference beam, also called copying beam, passes through the copying film previously laminated onto the master or onto an intermediate plate, which is generally a glass plate. The beam is reflectively diffracted by the master and passes through the copying film as so-called object beam, once more. The master is generally constructed of individual components which are used in order to achieve the requisite efficiencies in spectral reflection. The use of highly reflective masters is preferable, since it enables the production of copies with maximum efficiency, i.e., with minimal light power for the copying beam, down to a theoretical limit of 50:50 for the intensity ratio of object beam to reference beam. It must nonetheless be taken into account that, depending on the photochemical processes which take place in the copying film, on the incident angles of the laser beam and on the reconstruction angles of the master, intensity ratios less than 50:50, for example 30:70 to 10:90, may be required in order to achieve the desired diffraction efficiency and hence brightness in the copy.

At this point, then, a copy of the master hologram has been introduced into the copying film. All that is left to do at this stage is to fix the copy. To this end, following a delay time from the end of laser exposure, the so-called dark reaction time, which is 1-60 s, preferably 8-12 s, more preferably 10 s, lamp exposure with actinic radiation is started in order that the copying film may be cured, fixed and bleached. The spectral range of the lamp used is preferably 100-1000 nm, more preferably 200-800 nm, still more preferably 250-550 nm. This entire process is realizable in a reel-to-reel process. The master may be designed in sheeetlike form as a plate for step-and-repeat or be drum mounted for a continuous replication.

Replication may utilize laser wavelengths and light power ranges that are non-standard and thus represent an additional obstacle to the counterfeiter, provided the hologram is in line with the typical set of characteristics and is actually copiable at its original exposure wavelength. [When used in contact copying off the master, the photopolymer of the present invention gives a wavelength offset which is less than the spectral full width at half maximum values of the hologram. A numerical example: a volume Bragg grating having a maximized diffraction efficiency of 1 in reflection has a spectral width of 16.5 nm (for an assumed 15 nm film thickness and an index modulation of 0.036). The 16.5 nm are greater than the typical wavelength offset of 3-7 nm.]

Alternatively, color tuning methods can be used to shift the reconstruction wavelength into a more copyproof region.

What follows is a description of the application of the printed device to the security element by using a conventional inkjet printing technology.

The individual printed image here is integrated into the security concept of the security element according to the present invention such that there is efficient protection against forgery, mass copying and passing off. This is done by using a liquid printing ink, a printing substrate compatible therewith and suitable printing parameters aligned with each other such that the liquid ink will penetrate sufficiently far into the substrate and thereby delivers the required protection against manipulations. Possible attempts at manipulation include, for example, wiping off, erasing, scratching or comparable mechanical attacks, including in conjunction with incipient chemical dissolving, attempted bleaching or, in particular, washoff of the printed image.

The depth to which the liquid ink penetrates into the substrate is preferably by 20-100% and more preferably by 50-100%. The depth of penetration is experimentally detei minable using a confocal laser microscope for example.

The liquid ink of the present invention was introduced into a cartridge which is part of the printing head. The substrate to be printed has a temperature close to room temperature (16° C. to 25° C.), preferably a temperature of 22° C.

The distance of the printing nozzles from the printing substrate and also further printing parameters such as scan speed, nozzle spacing, nozzle diameter determine the resolution and quality of the printed image. All common models of inkjet printers are usable.

The printed image and the primary security features of the security element according to the present invention will now be described.

Reliable visual checkability requires that not just the trained observer, e.g., the security printer or the merchant, but also the untrained user be enabled to identify the security element quickly and ideally unequivocally as the original; this needs clear visual information which can only be copied/duplicated at prohibitive cost and inconvenience.

The requirement of decorativeness is derived from the requirements that, although the label should be conspicuous, it should nonetheless and at the same time augment the design of the product or of its packaging. The end product should be visually enhanced. The integration of the label into the product design shall be balanced, in particular as far as the color and the transparency of the label is concerned. A colored 2D/3D or 3D photopolymer hologram meets these requirements. Owing to its coloredness but also its transparency outside the hologram image and/or outside its reconstruction angle region, it is combinable with printed motifs, for example the printed image on a collapsible box.

The individual information should be in the form of visible information or at least comprise visible elements. Depending on the intended use it may be for example any desired serial code, or a machine-readable (alpha)numeric code, bar code, QR data matrix code, or in the form of individual product information. In the case of personalized products, this information may have relevance to the person or collective to be identified. Devices of this type are known from other printing processes, for example from the offset printing of bar codes on paper and plastic or from the laser engraving in polycarbonate or PVC-based security documents, see for example the US 20090251749 application. Therefore, the requirement of machine readability/authentication of the printed devices according to the present invention is the same as stipulated by state-of-the-art reading equipment makers. It relates primarily to resolution, contrast and color.

It is a further security feature that the printed liquid ink prevents illegal reproductions of the holographic security device by contact copying. This can be accomplished on the basis of three different mechanisms:
1. The liquid ink absorbs the copying beam, which is supposed to reach the recording medium as object beam, to a partial extent and thereby prevents the hologram replication attaining sufficient intensity, and/or alters the object-to-reference beam ratio to a crucial extent. As a consequence, the diffraction efficiency of the hologram replication becomes too low in the region of overlap with the printed image, and copy and original can be distinguished.
2. The liquid ink induces light scattering which reduces the diffraction efficiency of the copy. The explanation is that the efficiently usable dynamic range of the photopolymer is reduced by the intermodulation noise which is caused by the liquid ink and which in turn writes competing secondary holograms (“ghost holograms”).
3. The liquid ink prevents contact copies by bathochromically shifting the reconstruction color of the hologram image continuously away from the particular initial color (which varies locally in the colored imaging hologram of the present invention) via the color tuning effect. It was observed in relation to the printed liquid inks of the present invention that the intensity of the spectral shift, as measured in wavelengths [nm], is more intensive in the center of a printed field of constant dot density than at its edge. A 1:1 reproduction of the continuous spectrum of the colors is very costly and inconvenient, since it would require many reproduction lasers with narrow wavelength spacing.

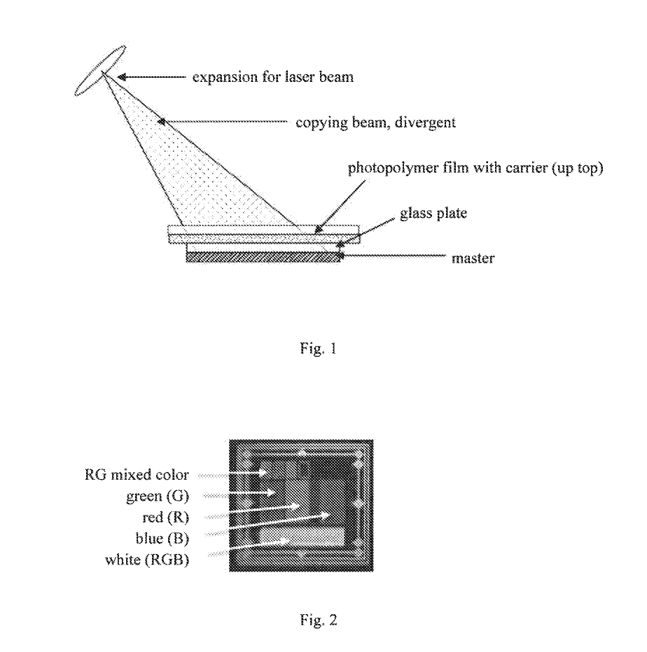

The invention will now be more particularly elucidated by means of examples and FIGS. 1 to 7, where

FIG. 1 shows a schematic exposure setup,

FIG. 2 shows a photographic picture of a volume hologram master in the viewing direction perpendicularly to the master plate.

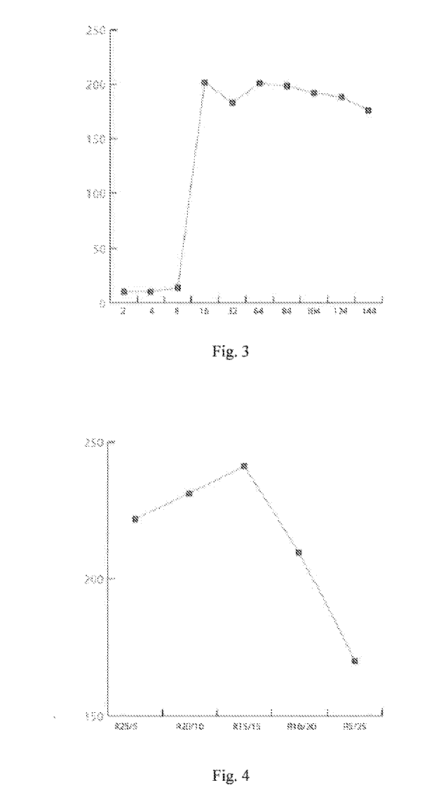

FIG. 3 shows measured results charted as intensity values (red) [arbitrary units] versus exposure time [s],

FIG. 4 shows measured results charted as intensity values (red) [arbitrary units] versus exposure sequence [s/s],

FIG. 5 shows measured results charted as RGB intensity values [arbitrary units] versus exposure time [s] under simultaneous RGB exposure,

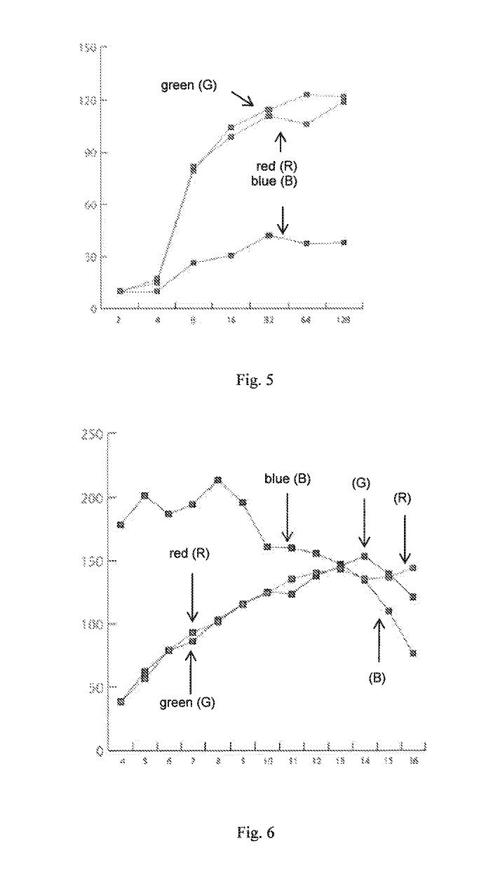

FIG. 6 shows measured results charted as RGB intensity values [arbitrary units] versus exposure time [s], and also

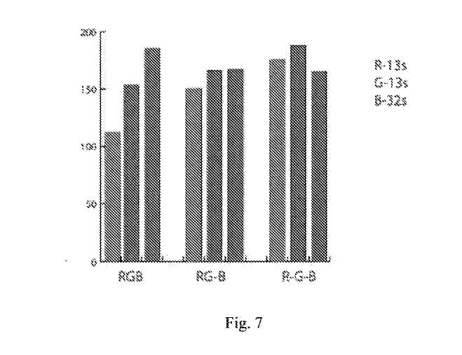

FIG. 7 shows measured results charted as RGB intensity values [arbitrary units] under different exposure sequences,

### EXAMPLE 1

### Contact Exposures of Master, Optimizing the Exposure Sequence

FIG. 1 shows the schematic exposure setup used for optimizing the RGB laser exposure sequence. The photopolymer used was Bayfol® HX 101 from Bayer MaterialScience AG (Leverkusen, Germany). The photopolymer layer is 16 μm in thickness and has a 36 μm PET carrier foil. A concealer was removed from the other side of the photopolymer layer and the photopolymer was laminated with its free side onto the spacer glass in front of the master hologram. The PET foil faces the expanded laser beam(s). The laser beam is white since it is made up of overlapping laser beams in the colors red, green and blue. Individual laser beams may be blanked off to create mixed colors or monochromatic illumination. The master, which is based on silver halide, reconstructs at the laser wavelengths used and creates the object beam in the photopolymer. The master hologram used was a colored volume hologram which has two-dimensional, monochromatically reflective scattering image areas as well as areas with additive colors, as can be seen in FIG. 2. The overall setup is situated in a dark laboratory.

### EXAMPLE 1a

### Creating a Monochromatically Red Hologram Copy

The red 633 nm laser is directed at an intensity of 1.5 mW/cm² onto the photopolymer film. Exposure time was varied in the exposure series, amounting to 2, 4, 8, 16, 32, 64, 84, 104, 124 and 144 s for the respective samples to be exposed. The result found was that visible, bright holograms form at an introradiated energy density of about 12 mJ/cm² or more. Considered on the energy density scale, brightness reaches a saturation value at above 12 mJ/cm² before coming back down slightly thereafter. The measured results are charted in FIG. 3.

### EXAMPLE 1b

### Discontinuous Exposure to Red Laser

The exposure process is slightly modified: exposure to the red 633 nm laser is interrupted for 1 min. Overall exposure time, i.e., the introradiated energy dose, was kept constant. The exposure sequences were as follows:
a) 25 s; pause for 1 min; a further 5 s
b) 20 s; pause for 1 min; a further 10 s
c) 15 s; pause for 1 min; a further 15 s
d) 10 s; pause for 1 min; a further 20 s
e) 5 s; pause for 1 min; a further 25 s

The result found was that visible, bright holograms are formed in all cases, as can be seen in FIG. 4. Exposure sequence (c), see R15/15 in FIG. 4, delivers the brightest hologram; nonetheless, the differences are small. The experiment also shows that the photopolymer can also be exposed in temporal sequences.

### EXAMPLE 1c

### Simultaneous Three-Color Exposure

The contact exposures were carried out with overlapping laser beams in the colors red (633 nm wavelength), green (561 nm) and blue (491 nm) concurrently for 2, 4, 8, 16, 32, 64 and 128 s exposure time at 6 mJ/cm² dose per color. The brightnesses of the individual spectral components of the hologram copy were measured. All three colors of the master were successfully reproduced. Blue was comparatively weaker than red and green, as FIG. 5 shows, but this can be optimized by adjusting the exposure parameters.

### EXAMPLE 1d

### Optimizing the Color Balance for Use in Practice

These experiments verify that successive as well as simultaneous RGB exposures lead to copies having bright RGB picture holograms. Differences in absolute and relative color brightness are observed depending on the choice of exposure settings. Adjustment of the color balance to the desired value (e.g., achieving a target whiteness from selected white light sources for the reconstruction) is thus possible.

The following example (FIG. 6) demonstrates how the point of equally intensive individual colors is attainable and how the intensity curves depend on the exposure time. A blue exposure of 32 s duration follows in each case a simultaneous RG exposure of 4 to 16 s duration. The curves have a point of intersection at 13 s RG exposure time.

The histograms in FIG. 7 show that the whiteness is adjustable via the form of temporal grouping for the laser beams by choosing the exposure times in accordance with the point of intersection in FIG. 6. In FIG. 7, the left-hand group corresponds to simultaneous exposure to three laser colors, the middle group corresponds to an RG exposure with subsequent B- exposure and the right-hand group corresponds to a sequential RGB exposure.

### EXAMPLE 2

### Preparation of Dyes

### Colorless Ammonium Salts

### EXAMPLE A-1

### Benzyldimethylhexadecylammonium bis(2-ethylhexyl)sulfosuccinate

3.00 g of sodium bis(2-ethylhexyl)sulfosuccinate and 2.79 g of benzyldimethylhexadecylammonium chloride hydrate were stirred in 30 ml of ethyl acetate at room temperature for 3 h. The reaction mixture was filtered through a pleated filter and the filtrate was desolventized. The residue was dried at 50° C. in vacuo to leave 5.03 g (95.3% of theory) of a colorless honey-like substance of the formula

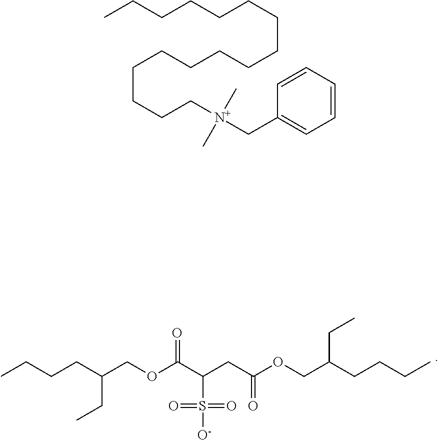 [SMILES: CCCCCCCCCCCCCCCC[N+](C)(C)Cc1ccccc1 ; CCCCC(CC)COC(=O)CC(C(=O)OCC(CC)CCCC)S(=O)(=O)[O-]]

T_{G}=−52° C.

characteristic signals in ¹H NMR in CDCl₃: δ=4.70 (s, 2H, C₆H₅—CH₂—), 4.05 (dd, 1H, CH—SO₃⁻), 3.95 (d, 4H, —O—CH₂—), 3.10 (s, 6H, (CH₃)₂N⁺).

### EXAMPLE A-2

### N,N,N,N′,N′N′Hexabutylhexamethyleriediammonium bis(bis(2-ethylhexyl)sulfosoceinate)

1.10 g of N,N,N N′N′N′-hexabutylhexamethylenediammoniurn dihydroxide used as 20 weight percent aqueous solution was adjusted with 10 percent hydrochloric acid to pH=7. The solution was evaporated to dryness in vacuo. The colorless crystalline mass was finely crushed and suspended in 25 ml of ethyl acetate. 2.00 g of sodium bis(2-ethylhexylsulfosuccinate were added. The mixture was stirred at room temperature for 3 h, in the course of which everything dissolved bar a fine suspension of salt. The reaction mixture was filtered through a pleated filter. The filtrate was desolventized. The residue was dried at 50° C. in vacuo to leave 2.16 g (74.0% of theory) of a colorless honey-like substance of the formula

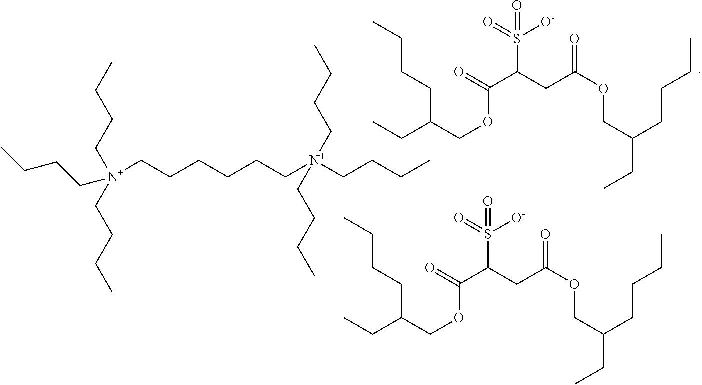 [SMILES: CCCC[N+](CCCC)(CCCC)CCCCCC[N+](CCCC)(CCCC)CCCC ; CCCCC(CC)COC(=O)CC(C(=O)OCC(CC)CCCC)S(=O)(=O)[O-] ; CCCCC(CC)COC(=O)CC(C(=O)OCC(CC)CCCC)S(=O)(=O)[O-]]

T_{G}=−45° C.

characteristic signals in ¹HNMr in CDCl₃:b δ=4.05 (dd, 2×1 H, CH—SO₃⁻), 3.95 (d, 2×4H,—O—CH₂—), 1.00 (s, 18H, CH₃—CH₂CH₂CH₂—N³⁰).

### EXAMPLE A-3

### Benzyl bis(2-hydroxyethyl)hexadecylammonium bis(2-ethylhexyl)sulfosuccinate

7.60 g of N-lauryldiethanolamine and 3.52 g of benzyl chloride were stirred at 65-70° C. for 13 h under agitation. 10 ml of cyclohexane were added to the hot mixture. After cooling down to room temperature under agitation, the suspension was filtered off with suction and washed with 5 ml of cyclohexane. Drying at 50° C. in vacuo left 9.95 g (89.5% of theory) of a colorless powder of the formula.

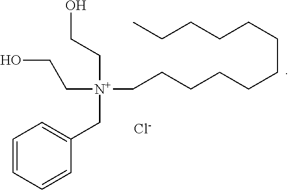 [SMILES: CCCCCCCCCCCC[N+](CCO)(CCO)Cc1ccccc1 ; [Cl-]]

2.70 g of this salt were stirred with 3.00 g of sodium bis(2-ethylhexyl)sulfosuccinate in a mixture of 50 ml of ethyl acetate and 50 ml of water at 40° C. for 2 h. The aqueous phase was separated off in a separating funnel and the organic phase was washed three times with 10 ml of water. Finally, the organic phase was dried with magnesium sulfate and evaporated to give 3.75 g (70.6% of theory) of a slightly yellowish viscous oil of the formula

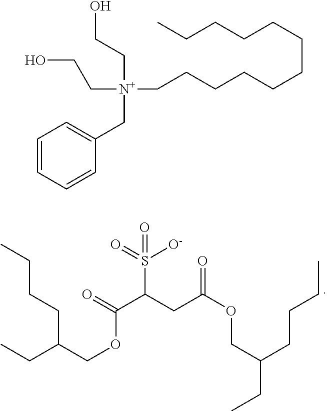 [SMILES: CCCCCCCCCCCC[N+](CCO)(CCO)Cc1ccccc1 ; CCCCC(CC)COC(=O)CC(C(=O)OCC(CC)CCCC)S(=O)(=O)[O-]]

T_{G}=−58° C.

characteristic signals in ¹H NMr in CDCl₃: δ=4.77 (s, 2H C₆H₅—CH₂—), 4.15 (d, 4H, —O—CH₂—),4.08 (dd, 1H, CH—SO₃⁻), 4.00, 3.92, 3.55, 3.52 (every m, every 2H, (HOCH₂—CH₂)₂—N⁺).

The following colorless salts were obtained in a similar manner:

| Ex- | | | | |
|---|---|---|---|---|
| ample | Cation | Anion | Form and yield | Solubility |
| A-4 | trioctylmethyl- | bis(2- | honey, | in EtOAc or |
| | ammonium | ethylhexyl)- | T_{G} = −77° C., | BuOAc |
| | | sulfosuccinate | 64.4% | |
| A-5 | octadecyl- | bis(2- | honey, | in EtOAc or |
| | trimethyl- | ethylhexyl)- | T_{G} = −50° C., | BuOAc |
| | ammonium | sulfosuccinate | 82.4% | |
| A-6 | dioctadecyl- | bis(2- | wax, T_{G}: not | in EtOAc or |
| | dimethyl- | ethylhexyl)- | measurable, | BuOAc |
| | ammonium | sulfosuccinate | 75.6% | |
| A-7 | octadecyl- | Turkey red oil | wax, | in ethanol, |
| | trimethyl- | | T_{G} = −54° C., | BuOAc |
| | ammonium | | 82.5%, | |

Lanthanide Complexes:

### EXAMPLE B1

### Tetrabutvlammonium salt of Europium Complex with 4-thienyl-1,1,1-trifluoro-butane-2,4-dione

The method of DE 69103448 was repeated except that tetrabutylammonium hydroxide was substituted for tetramethylainrnoniunn hydroxide. The europium complex of the formula

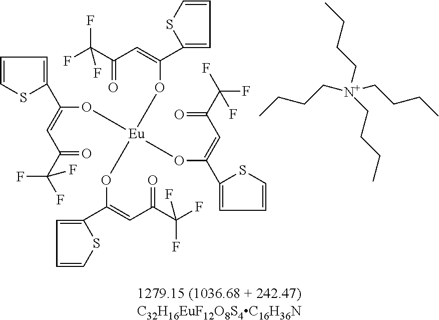

was obtained in 61.2% (of theory) yield as colorless powder.

Cationic Dyes:

### EXAMPLE C-1

### Basic Blue 3-(bis(2-ethylhexyl)sulfosuccinate)

15.0 g of sodium bis(2-ethylhexyl)sulfosuccinate (obtained from Aldrich in 2010) were dissolved in 350 ml of water at 50° C. 24.5 g of the dye of the formula

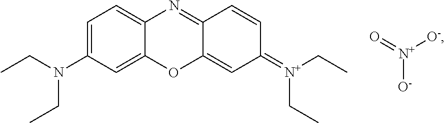

(Basic Blue 3), as 53 wt % product, and 220 ml of butyl acetate were added and stirred in at 50° C. for 4 h. The aqueous phase was separated off and the organic phase was stirred up three times with 50 ml of fresh water at 50° C. Finally, the aqueous phase was separated off each time, the last time at room temperature. The deep blue organic phase was dried with anhydrous magnesium sulfate, filtered and freed of residual water by azeotropic distillation at 150 mbar. Anhydrous butyl acetate was added to finally obtain 250 g of a deep blue solution which was 9.68 wt % strength in respect of the dye of the formula

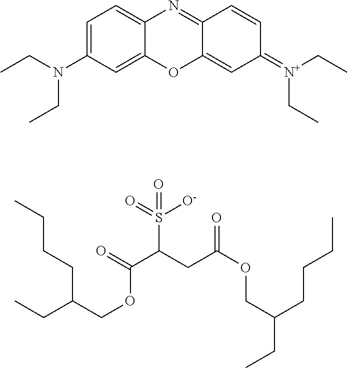

(96.4% of theory),

λₘₐₓ in methanol: 643 nm.

The solution was evaporated to leave 24.2 g of a deep blue glass which gradually crystallizes in the form of goldenly lustrous prisms. These were successfully converted, for example, back into 20 wt % solutions in butanone or 7:3 ethyl acetate/butanone.

The following dyes were obtained in a similar manner:

| Example | Cation | Anion | λₘₐₓ | Solubility |
|---|---|---|---|---|
| C-2 | 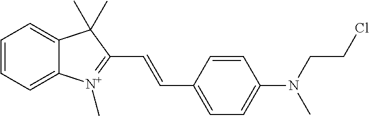 [SMILES: CN(CCCl)c1ccc(/C=C/C2=[N+](C)c3ccccc3C2(C)C)cc1] | 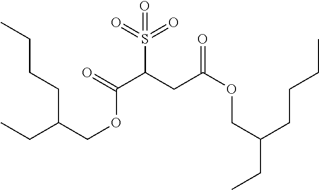 | 527 nm | in EtOAc, BuOAc |
| C-3 | 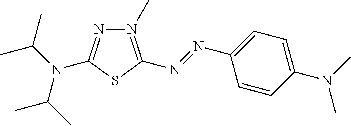 [SMILES: CC(C)N(c1n[n+](C)c(/N=N/c2ccc(N(C)C)cc2)s1)C(C)C] | 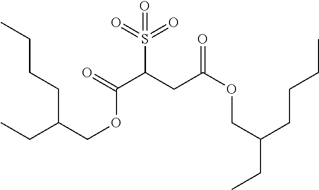 | 600 nm | in EtOAc |
| C-4 | 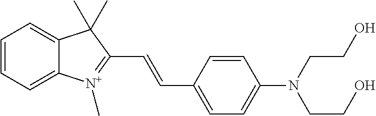 [SMILES: C[N+]1=C(/C=C/c2ccc(N(CCO)CCO)cc2)C(C)(C)c2ccccc21] | 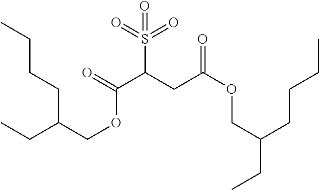 | 552 nm | in EtOAc, BuOAc |
| C-5 | 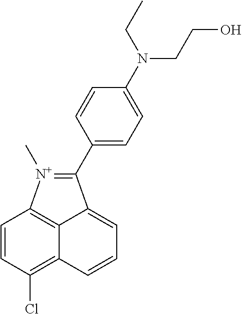 [SMILES: CCN(CCO)c1ccc(C2=[N+](C)c3ccc(Cl)c4cccc2c34)cc1] | 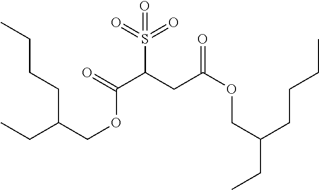 | 613 nm | in EtOAc, BuOAc |

Anionic Dyes:

### EXAMPLE D-1

### Acid Red 82 methyltrioctylammonium salt

The solutions of 1.64 g of Acid Red 82 in 35 ml of water and of 2.43 g of methyltrioctylatnmonium chloride in 30 ml of butyl acetate were mixed and the mixture was stirred at room temperature for 3 h. The aqueous phase was separated off in a separating funnel and the deep red organic phase was washed five times with 20 ml of water. Finally, the organic phase was dried with magnesium sulfate and evaporated to dryness. The residue was dried at 50° C. in vacuo to leave 3.60 g (96.7% of theory) of a red crystalline powder of the formula

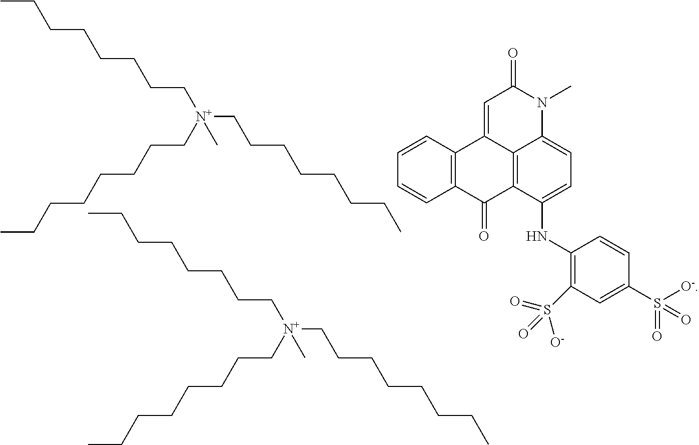

λₘₐₓ 543, 520 (sh) nm (13460).

A stable solution can be prepared in a mixture of 45 ml of butyl acetate and 20 ml of butanone.

### EXAMPLE 3

### Mixing the Liquid Printing Inks

Various colorless, colored and fluorescent dyes were dissolved in butyl acetate and diluted.

Listing of Mixes:

| Ex- | Solvent | Dye usage, effective | | |
|---|---|---|---|---|
| ample | Amount [g] | wt % | Amount [g] | Observation |
| A-4 | 39.800 | 100.0% | 0.200 | colorless |
| A-5 | 39.600 | 50.0% | 0.400 | colorless |
| A-1 | 39.000 | 20.0% | 1.000 | colorless |
| A-6 | 39.149 | 23.5% | 0.851 | colorless |
| A-7 | 38.182 | 11.0% | 1.818 | colorless |
| A-2 | 39.412 | 34.0% | 0.588 | colorless |
| A-3 | 39.800 | 100.0% | 0.200 | colorless |
| C-2 | 38.305 | 11.8% | 1.695 | pink |
| C-1 | 38.425 | 12.7% | 1.575 | turquoise |
| C-3 | 38.802 | 16.7% | 1.198 | blue |
| C-4 | 38.198 | 11.1% | 1.802 | violet |
| C-5 | 38.913 | 18.4% | 1.087 | blue |
| D-1 | 36.923 | 6.5% | 3.077 | pink |
| B-1 | 39.800 | 100.0% | 0.200 | fluorescent |

### EXAMPLE 4

### Pipetting the Liquid Inks onto Hologram Substrate

The hologram substrate selected was the RGB-capable photopolymer Bayfol® HX 101 (manufacturer: Bayer MaterialScience) exposed beforehand to a green 532 nm laser in a volume-holographic contact-copying process. The imaged hologram was in effect a mirror with a diffuse green reflection. The hologram in the photopolymer was subsequently fixed by the UV/VIS light of an iron-doped mercury lamp. The liquid printing inks consisting of component 1 (dye) and component 2 (butyl acetate solvent) were Eppendorf pipetted onto the photopolymer layer in amounts of 20 μl(preliminary tests) and 2 μl (final series of measurements) so as to form a standing droplet. After application, the droplet was wiped off within a few seconds.

The remaining ink then penetrated sufficiently far into the substrate to create color tuning in the hologram, The liquid inks listed hereinbelow by way of example achieved bathochromic shifts in the hologram (“color tuning”) from green via yellow, red as far as infrared. The color shift was strongest in the center of the droplet, i.e., it was only there that infrared was reached, and the strength of the color shift decreased in the outward direction.

### EXAMPLE 5

### Printing the Liquid Inks

The printing tests were carried out using an LPSO inkjet printer from PixDro B.V., where replenishing the printing cartridge (part of the miniature ink supply system) is possible. It is therefore easily possible to adjust the printer settings and to optimize them for printed image quality. Cartridge and ink supply system are robust with regard to many chemicals, making it possible to use various solvents for the ink and for the cleaning procedure.

The electric printing head voltages U=20 V, 40 V, 60 V were tested. The best result was obtained with voltages in the range from 40 to 60 V. The following parameters were chosen for the experiments: printing head voltage 60 V, printing head temperature 28° C. (substrate at room temperature).

The setting used for the gas pressure control system ensured availability not only of sufficient negative pressure for the experiment (setting: 17 mbar) but also of sufficient positive pressure for rapidly exchanging the ink.

Various distances for the printing head from the substrate table were tested: Z=2 mm, 1 mm and 0.5 mm, see also “Motion System/Z-axis” in the operating manual. The various settings in the Z-axis did not have any determinative influence on the quality of the printed image. The printing tests were therefore carried out with the Z=2 mm setting.

The PixDro signature offered in the software was chosen as the digital image to be printed. The image with lines of differing size (0.25 mm to 1 mm) and round dots (about 2 mm in diameter) was highly suitable for investigating the color tuning effect.

The printing head used has 128 nozzles. Resolution, droplet rate, time-of-flight and other quality-relevant parameters were optimized for any one ink, although not checked within the series of measurements. Nor were the inks checked/selected for maximum attainable resolution. Regarding crispness after printing and the stability of the printed image in 3 months' storage, no significant differences were observed between the liquid inks printed: the printed images looked clean in each case.

The substrates used were paper (in preliminary tests only) and the holographic photopolymer of Example 4.

Some of the mixed inks were additionally admixed with further butyl acetate for dilution in order to vary the contrasts.

Tests were also carried out whereby two ink mixes were mixed with each other and the mixture was printed, indicated in the table hereinbelow by two paired rows.

The interaction between ink and hologram was observed and assessed using the eye under ceiling illumination.

The following inventive inks and substrates were used and the following results were obtained; butyl acetate was used as solvent in all cases:

| | | | Evaluation of printed image | | |
|---|---|---|---|---|---|
| | | | Color/ | | |
| Ex- | Sol- | Ink, conc. | resolu- | | |
| ample | vent | wt % | tion | Contrast | Hologram |
| C-2 | BuAc | 0.10% | pink | weak | angle detuning, |
| | | | | | weak effect |
| C-1 | BuAc | 0.10% | turquoise | weak | angle detuning, |
| | | | | | weak effect |
| C-3 | BuAc | 0.10% | blue | weak | angle detuning, |
| | | | | | weak effect |
| C-4 | BuAc | 0.10% | violet | weak | angle detuning, |
| | | | | | weak effect |
| A-4 | BuAc | 0.50% | colorless | | angle detuning, |
| | | | | | weak |
| A-5 | BuAc | 0.50% | colorless | | angle detuning, |
| | | | | | stronger |
| A-1 | BuAc | 0.50% | colorless | | angle detuning, |
| | | | | | stronger |
| A-6 | BuAc | 0.50% | colorless | | angle detuning, |
| | | | | | weak effect |
| A-7 | BuAc | 0.50% | colorless | | angle detuning, |
| | | | | | stronger |
| A-2 | BuAc | 0.50% | colorless | | angle detuning, |
| | | | | | weak |
| A-3 | BuAc | 0.50% | colorless | | angle detuning, |
| | | | | | stronger |
| C-2 | BuAc | 0.50% | pink | good | angle detuning, |
| | | | | | weak effect |
| C-1 | BuAc | 0.50% | turquoise | good | angle detuning, |
| | | | | | weak effect |
| C-3 | BuAc | 0.50% | blue | good | angle + |
| | | | | | color det. |
| | | | | | (from green |
| | | | | | to red) |
| C-4 | BuAc | 0.50% | violet | good | angle detuning, |
| | | | | | weak effect |
| C-5 | BuAc | 0.50% | blue | good | angle detuning, |
| | | | | | very weak |
| D-1 | BuAc | 0.50% | pink | good | angle detuning, |
| | | | | | very weak |
| B-1 | BuAc | 0.50% | fluorescent | good, | |
| | | | | UV lamp | |
| C-3 | BuAc | 0.25% | blue, good | still good | angle detuning |
| A-5 | BuAc | 0.25% | | | (best value |
| | | | | | of all ink |
| | | | | | mixtures) |
| B-1 | BuAc | 1.00% | pink, good | weak + | angle detuning |
| C-2 | BuAc | 0.50% | | fluorescent | |
| B-1 | BuAc | 1.00% | colorless | fluorescent | angle detuning |
| A-5 | BuAc | 0.50% | | | |
| B-1 | BuAc | 1.00% | colorless | fluorescent | angle detuning |
| A-2 | BuAc | 0.50% | | | |
| A-2 | BuAc | 0.50% | pink, good | weak. | angle detuning |
| C-2 | BuAc | 0.50% | | | |
| A-2 | BuAc | 0.50% | turquoise, | weak | angle detuning |
| C-1 | BuAc | 0.50% | good | | |

Angle detuning is used to identify hologram regions having altered illumination and/or viewing angles. The microscopie cause is believed to be spatial swelling of holographic grating structures resulting in substantial alteration of grating vectors especially at the boundary of printed contours, i.e., the gratings sag. The result is that the holograms light up under different angles in the region of the printed image.

These kinds of changes are very easy to see with the naked eye because the diffracted light is marked by high contrast.

The angle tuning effect was stronger than the color tuning effect, which varied in a barely discernible manner as between grass green and lime green. Only in the case of ink C-3 was color tuning observable with a distinct change in color in the direction of orange-red. Both the effects, angle and color change, are equally useful when inkjet printing is used for the forgeryproof marking of holograms,

### EXAMPLE 6

### Thermal Aging of Printed Substrates

Printed substrates from Example 5 were subjected to aging at elevated temperature. One sample at a time was laminated with its photopolymer side facing down onto a 1 mm thick glass carrier and placed in this orientation between the heating platens of an FP82 HT heating stage (FP 90 Controller from Mettler Toledo).

Colored inks were tested only.

The following temperature profile was chosen: starting temperature room temperature (22° C.); heating rate >5 K/min; target temperature 85° C.; duration of isothermal storage: 30 min; subsequent cooling down in air.

Examples of investigated inks: C-1, C-2, C-3, C-4, C-5 and D-1.

Result: No visible change in color strength, color value and resolution of printed image. All ink-substrate combinations proved thermally stable under these conditions.

1.-17. (canceled)

18. A method of producing a security element comprising a holographic layer containing a hologram, comprising at least the steps of
a) providing the holographic layer;
b) exposing the holographic layer at least sectionwise via a master hologram to produce a hologram copy in the holographic layer;
c) printing the holographic layer at least sectionwise with an ink to form a printed device, wherein the ink comprises a melt of a dye or of a colorless component or a solvent and a dye or colorless component dissolved therein;
d) fixing the exposed holographic layer to produce the hologram in the holographic layer, wherein the printed device and the hologram are arranged in the holographic layer such that the printed device and the hologram overlap sectionwise at least.

19. The method as claimed in claim 18, wherein the printed device is formed before and/or after the production of the hologram copy and/or the fixing of the exposed holographic layer.

20. The method as claimed in claim 18, wherein the ink does not contain any constituents that are insoluble in the solvent, and/or in that the printing is effected via inkjet printing.

21. The method as claimed in claim 18, wherein the dye is a salt-type dye.

22. The method as claimed in claim 18, wherein the colorless component is a salt-type substance.

23. The method as claimed in claim 18, wherein the dye and/or the colorless component migrates into the holographic layer.

24. The method as claimed in claim 23, wherein the reconstruction color of the hologram, its diffraction efficiency and/or reconstruction angle are irreversibly altered by the dye which migrates into the holographic layer.

25. The method as claimed in claim 18, wherein the dye reflects white light in the visible wavelength range.

26. The method as claimed in claim 18, wherein the holographic layer comprises a photopolymer material and/or the holographic layer is on a carrier.

27. The method as claimed in claim 18, wherein the hologram is formed by a volume hologram, sectionwise at least.

28. The method as claimed in claim 18, wherein the hologram reconstructs light of at least two different wavelengths in the visible spectrum, wherein the different wavelengths are more particularly at least 10 nm apart.

29. The method as claimed in claim 18, wherein the hologram area overprinted by the printed device comprises from 5 to 95% of the entire area of the hologram, and/or wherein the printed device projects beyond the hologram on one side at least.

30. The method as claimed in claim 18, wherein the printed device is an image, a pattern, an alphanumeric code, a 2D or 3D bar code, a machine-readable code, or a biometric feature.

31. A security element obtained by the method as claimed in claim 18.

32. A document, a certificate or document of value, a banknote, an ID card, a high security access card, a tax seal, an electronic ticket, an electronic card, a credit card, a cashcard or a product package or product label for consumer durables, industrial goods and consumable goods, endowed with a security element as claimed in claim 31.

33. A method comprising using an ink to improve the anti-counterfeit security of a hologram wherein the ink comprises a melt of a dye or of a colorless component or a solvent and a dye or colorless component dissolved therein.

34. A compound of the formula 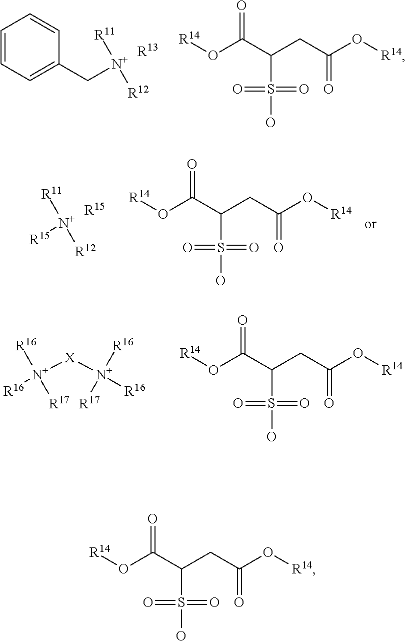
wherein
R¹¹ and R¹² are each independently methyl, ethyl, propyl, butyl, hydroxyethyl or cyanoethyl,
R¹³ is C₁₆- to C₂₂-alkyl or is C₁₀- to C₂₂-alkyl when R¹ and R² are not both methyl,
R¹⁴ is optionally branched C₆- to C₁₂ alkyl,
R¹⁵ is C₁₂- to C₂₂-alkyl,
R¹⁶ and R¹⁷ are each independently methyl, ethyl, propyl or butyl,
R¹⁷ is additionally benzyl,
X is a —(CH₂)ₙ— bridge, and
n is an integer from 4 to 10.

35. The method as claimed in claim 21, wherein the dye is a cationic dyes selected from the group consisting of acridine dyes, xanthene dyes, thioxanthene dyes, phenazine dyes, phenoxazine dyes, phenothiazine dyes, coumarin dyes, tri(het)arylmethane dyes, mono-, di-, tri-, tetra- and pentamethinecyanine dyes, hemicyanine dyes, diazahemicyanine dyes, zeromethine dyes, streptocyanine dyes, externally cationic merocyanine dyes, externally cationic neutrocyanine dyes, externally cationic phthalocyanine dyes, externally cationic anthraquinone dyes, and externally cationic azo dyes, or an anionic dye selected from the group consisting of oxonols, di- and trihydroxy-triarylmethane dyes, merocyanine, neutrocyanine, coumarin, anthraquinone, anthrapyridone, dioxazine, mono-, dis- and trisazo dyes having at least one sulfo group, acridine, xanthene, thioxanthene, phenazine, phenoxazine, phenothiazine, tri(het)arylmethane dyes, phthalocyanines and azo metal complexes bearing at least one sulfo group, and also mixtures thereof.
